(19) United States
(12) Patent Application Publication
(10) Pub. No.: US 2026/0234091 A1
(43) Pub. Date: Aug. 13, 2026

(54) FRAGRANCE AND FLAVOR COMPOSITIONS COMPRISING ADAMANTANE DERIVATIVES

(71) Applicant: OSMO LABS, PBC., New York, NY (US)

(72) Inventors: Alexander WILTSCHKO, Somerville, MA (US); Brendan LEHNERT, Cambridge, MA (US); Chase BUCHHOLZ, Chicago, IL (US); Jacob YASONIK, Cambridge, MA (US); Wei QIAN, Cambridge, MA (US); Bradley B. GILBERT, Milford, NJ (US); Andrew P. PATRON, Oceanside, CA (US)

(21) Appl. No.: 19/153,944

(22) PCT Filed: Feb. 6, 2024
(86) PCT No.: PCT/US2024/014675

§ 371 Date: Aug. 5, 2025

Related U.S. Application Data
(60) Provisional application No. 63/483,422, filed on Feb. 6, 2023.

(51) Int. Cl. C07C 35/37; C07C 43/18; C07C 69/608

(52) U.S. Cl. CPC C07C 35/37; C07C 43/18; C07C 69/608; C07C 2603/74

(57) ABSTRACT

The present application relates to 2-ethyl-2-adamantanol and related compounds, methods of making them, and methods of using them as flavor and fragrance ingredients in food, cosmetic, pharmaceutical, consumer, and other compositions and products.

### CROSS-REFERENCE TO RELATED APPLICATIONS

This application is an international application which claims priority to, and the benefit of, U.S. Provisional Application Ser. No. 63/483,422, filed on Feb. 6, 2023, the contents of which are hereby incorporated by reference in its entirety.

### FIELD OF THE APPLICATION

The present application relates to 2-methyl-2-adamantanol, 2-ethyl-2-adamantanol, and related compounds, methods of making them, and methods of using them as flavor and fragrance ingredients in food, cosmetic, pharmaceutical, consumer, and other compositions and products.

### BACKGROUND

Scent is an important factor used to produce a sense of anticipation, quality, palatability, and security to many consumer products. Flavor is particularly important for foodstuffs. Identifying effective aromas and flavors to impart in a product is an element that contributes to the success of the product, and is useful in product marketing, consumer satisfaction, and consumer retention. Patchouli, camphoraceous, fresh, aqueous, airy, citrus, and zesty smells may be particularly desirable for certain flavors and fragrances and may be used in toiletries, cosmetics, household cleaners, room sprays, laundry, and fine fragrance applications, such as in perfumes and toilet water, dental hygiene products (such as toothpastes and mouthwashes), orally administered medications, and food products.

Considerable work is performed by many scientists relating to identifying new substances which can be used, alone or in combinations, to impart to, or enhance, the aroma or flavor of various consumable materials, including, e.g., cosmetics, cleaners, and foodstuffs. While there may be some trends in the relationship between chemical structure and flavor or fragrance—such as common use of low molecular weight aldehydes and alcohols as flavors and fragrances—the precise aroma associated with a molecule is exceedingly difficult to predict. Small changes in structure, such a lengthening or shortening a functional group by just one carbon atom, can have profound and unexpected effects on a compound's flavor or fragrance profile. The art of flavor and fragrance prediction is still in its infancy.

Using a proprietary predictive method, the inventors have identified 2-methyl-2-adamantanol and 2-ethyl-2-admantanol, as new ingredients useful in flavors and fragrances. These compounds have not been previously identified as a flavor or fragrance ingredients, nor has their smell or odor been previously described.

This application describes the surprising and unexpected olfactive qualities of 2-methyl-2-adamantanol and 2-ethyl-2-admantanol, and analogs and derivatives thereof, and their use as fragrance and flavor ingredients, and potential applications thereof.

### BRIEF SUMMARY

In one aspect, the application relates to compounds of Formula I:

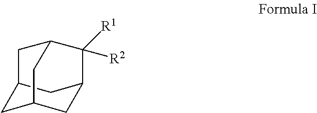

wherein:
   R¹ is selected from OH, OR³, O(CO)R³, CN, COOH, COOR³, CH₂OH, CH₂OR³, C(O)R³, CH(R⁴)OR³, CH(R⁴)OH, CHO, CH₂COOH, and CH₂COOR³;
   R² is H or optionally substituted C₁₋₆alkyl, C₂₋₆alkenyl, C₂₋₆alkynyl, C₃₋₆cycloalkyl; R³ and R⁴ are each independently selected from C₁₋₆alkyl, C₂₋₆alkenyl, C₂₋₆alkynyl, C₃₋₇cycloalkyl, C₃₋₇cycloalkenyl, heterocycloalkyl, aryl, and heteroaryl, each of which is optionally substituted by one or more groups as defined herein;
   wherein the adamantane core is optionally substituted by one or more groups as defined herein;
   optionally provided that the compound is not 2-methyl-2-adamantanol or 2-ethyl-2-admantanol.

In some embodiments, the compound of Formula I is 2-ethyl-2-admantanol or the compound of Formula I is 2-methyl-2-adamantanol.

In another aspect, the application relates to fragrance and flavor compositions comprising the compound of Formula I, optionally comprising one or more additives, additional fragrance or flavor ingredients, or a combination of additives and fragrance or flavor ingredients. In some embodiments, the application relates to fragrance and flavor compositions comprising 2-methyl-2-adamantanol or 2-ethyl-2-admantanol.

In another aspect, the application relates to products, such as consumer products, comprising such fragrance and flavor compositions comprising the compound of Formula I, e.g., products comprising 2-methyl-2-adamantanol or 2-ethyl-2-admantanol.

In another aspect, the present disclosure provides a method of making a compound of Formula I.

The details of one or more embodiments of the application are set forth in the accompanying description below. Unless defined otherwise, all technical and scientific terms used herein have the same meaning as commonly understood by one of ordinary skill in the art to which this application belongs. In the case of conflict, the present specification will control.

Other features and advantages of the application will be apparent from the following detailed description, examples, and claims.

### DETAILED DESCRIPTION

The inventors have unexpectedly found that 2-ethyl-2-admantanol has a unique aroma and flavor characterized by patchouli and camphoraceous aromas. The inventors have also unexpectedly found that 2-methyl-2-admantanol has a unique aroma and flavor characterized by camphor, citrus and zesty aromas. It has thus been determined to be useful in imparting and providing desirable aromas and/or flavors to the products to which it is added. Other Compounds of Formula I are expected to likewise have pleasant or desirable flavors and/or aromas. Such Compounds are therefore potentially useful for products where the inclusion of a pleasing fragrance or flavor is desired, including, but not limited to, perfumes, household products, laundry products, personal care products, cosmetics, dental hygiene products, orally administered medications, and food products. The Compounds of Formula I may be employed in varying amounts depending upon the specific fragrance or flavor product application, the nature and amount of other flavor or fragrance ingredients present, and the desired aroma and/or flavor of the product.

In a first aspect, the present disclosure provides a compound of Formula I (Compound 1):

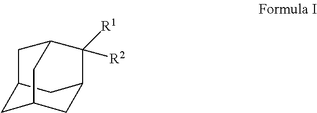

wherein:
   R¹ is selected from OH, OR³, O(CO)R³, CN, COOH, COOR³, CH₂OH, CH₂OR³, C(O)R³, CH(R⁴)OR³, CH(R⁴)OH, CHO, CH₂COOH, and CH₂COOR³;
   R² is H or optionally substituted C₁₋₆alkyl, C₂₋₆alkenyl, C₂₋₆alkynyl, or C₃₋₆cycloalkyl;
   R³ and R⁴ are each independently selected from C₁₋₆alkyl, C₂₋₆alkenyl, C₂₋₆alkynyl, C₃₋₇cycloalkyl, C₃₋₇cycloalkenyl, heterocycloalkyl, aryl, and heteroaryl, each of which is optionally substituted by one or more groups as defined herein;
   wherein the adamantane core is optionally substituted by one or more groups as defined herein;
optionally provided that the compound is not 2-methyl-2-adamantanol or 2-ethyl-2-admantanol.

In further embodiments, of the first aspect, the present disclosure provides:
1.1 Compound 1, wherein the compound is not 2-methyl-2-adamantanol or 2-ethyl-2-admantanol;
1.2 Compound 1, wherein the compound is not a compound wherein:
   (a) R¹ is OH and R² is H, methyl, ethyl, propyl, isopropyl, cyclopropyl, n-butyl, teit-butyl, cyclobutyl, sec-butyl, isobutyl, cyclopropylmethyl, cyclopentyl, 1-ethylpropyl, n-pentyl, n-hexyl, cyclohexyl, vinyl, allyl, 1-propenyl, isopropenyl,1-propynyl, 2-propynyl, hydroxymethyl, aminomethyl, iodomethyl, cyanomethyl, 2-hydroxyethyl, methoxymethyl, aminoethyl, 3-hydroxypropyl, or 2-oxoethyl;
   (b) R¹ is OCH₃ and R² is H, methyl, ethynyl, bromomethyl, aminomethyl, benzyl, allyl, 3-hydroxypropyl, isopropenyl, acetyl, carboxyl, CH₂COOH, CH₂CH₂COOH, 1-oxopropyl, or 3-oxobutyl;
   (c) R¹ is O(CO)R³ and R³ is C₁₋₂alkyl, and R² is methyl, ethyl, ethynyl, vinyl, carboxy, acetyl, cyanomethyl, bromomethyl, CH₂COOH, 3-oxobutyl, or 3-(dimethylamino)propyl;
   (d) R¹ is CH₂OH, and R² is methyl, aminomethyl, hydroxymethyl, allyl, 2-hydroxyethyl, vinyloxymethyl, (dimethylamino)methyl, 2-methoxyethyl, or CH₂O(CO)C(CH₃)═CH₂;
   (e) R¹ is CH₂OR³, R² is methyl, and R³ is CH2Cl, CH₂OCH═CH₃, CH₂O(CO)C(CH₃)═CH₂;
   (f) R¹ is CH₂OCH═CH₃, and R² is CH₂OCH═CH₃ or CH₂OH;
   (g) R¹ is COOH, and R² is methyl, 2-propenyl, aminomethyl, CH₂COOMe, CH₂CONHMe, CH₂CH₂COOH, CH₂CH₂CONHMe;
   (h) R¹ is COOMe, and R² is methyl, 2-propenyl, aminomethyl, dicyanomethyl, 2-methoxyethyl, CH₂COOMe, CH₂CONHMe, CH₂CH₂COOH, CH₂CH₂CONHMe, or 2-phenylethyl; and
   (i) R¹ is CN, and R² is methyl, hydroxymethyl, cyanomethyl, benzyl, 2-propenyl, n-butyl, aminohydroxymethyl, CH₂COOH, CH₂COOMe, CH₂COOEt, CH₂CH₂CH₂CO₂Et, 3-cyanopropyl, 2-methyl-2-buten-4-yl, 4-hydroxybutyl, 4-cyanobutyl, 4-chlorobutyl, 4-bromobutyl, or tetrazolylmethyl;
1.3 Compound 1, or any of 1.1-1.2, wherein R¹ is OH;
1.4 Compound 1, or any of 1.1-1.2, wherein R¹ CN, or CH₂OH, or COOH;
1.5 Compound 1, or any of 1.1-1.2, wherein R¹ is OR³;
1.6 Compound 1, or any of 1.1-1.2, wherein R¹ is O(CO)R³;
1.7 Compound 1, or any of 1.1-1.2, wherein R¹ is CH₂OH or CH₂OR³;
1.8 Compound 1, or any of 1.1-1.2, wherein R¹ is CH₂COOH or CH₂COOR³;
1.9 Compound 1, or any of 1.1-1.2, wherein R¹ is CH₂COOR³;
1.10 Compound 1, or any of 1.1-1.9, wherein R² is H;
1.11 Compound 1, or any of 1.1-1.9, wherein R² is not H;
1.12 Compound 1, or any of 1.1-1.9, wherein R² is unsubstituted C₁₋₆alkyl;
1.13 Compound 1, or any of 1.1-1.9, wherein R² is substituted C₁₋₆alkyl;
1.14 Compound 1.12 or 1.13, wherein said C₁₋₆alkyl is selected from methyl, ethyl, n-propyl, isopropyl, but-1-yl (n-butyl), but-2-yl (sec-butyl), 2-methyl-prop-1-yl (isobutyl), tert-butyl, pent-1-yl (n-pentyl), pent-2-yl (sec-pentyl), 3-methylbut-1-yl (isopentyl), pent-3-yl, and hex-1-yl (n-hexyl);
1.15 Compound 1, or any of 1.1-1.9, wherein R² is unsubstituted C₃₋₆cycloalkyl;
1.16 Compound 1, or any of 1.1-1.9, wherein R² is substituted C₃₋₆cycloalkyl;
1.17 Compound 1.15 or 1.16, wherein said C₃₋₆cycloalkyl is selected from cyclopropyl, cyclobutyl, cyclopentyl, and cyclohexyl;
1.18 Compound 1, or any of 1.5-1.9, wherein R³ is selected from optionally substituted C₁₋₆alkyl, optionally substituted C₂₋₆alkenyl, optionally substituted C₂₋₆alkynyl, optionally substituted C₃₋₆cycloalkyl, optionally substituted C₃₋₇cycloalkyl, optionally substituted C₃₋₇cycloalkenyl, optionally substituted heterocycloalkyl, optionally substituted aryl, and optionally substituted heteroaryl;
1.19 Compound 1, or any of 1.5-1.9, wherein R³ is selected from optionally substituted C₁₋₆alkyl, optionally substituted C₂₋₆alkenyl, optionally substituted C₂₋₆alkynyl, optionally substituted C₃₋₆cycloalkyl, optionally substituted C₃₋₇cycloalkyl, and optionally substituted C₃₋₇cycloalkenyl,
1.20 Compound 1, or any of 1.5-1.9, wherein R³ is selected from optionally substituted C₁₋₆alkyl, optionally substituted C₂₋₆alkenyl, optionally substituted C₂₋₆alkynyl, and optionally substituted C₃₋₆cycloalkyl;
1.21 Compound 1, or any of 1.5-1.9, wherein R³ is selected from optionally substituted C₁₋₆alkyl, optionally substituted C₂₋₆alkenyl, and optionally substituted C₂₋₆alkynyl;
1.22 Compound 1, or any of 1.5-1.9, wherein R³ is selected from optionally substituted C₁₋₆alkyl and optionally substituted C₂₋₆alkenyl;
1.23 Compound 1, or any of 1.5-1.9, wherein R³ is optionally substituted C₁₋₆alkyl;
1.24 Compound 1.23, wherein R³ is unsubstituted C₁₋₆alkyl;
1.25 Compound 1.23, wherein R³ is substituted C₁₋₆alkyl;
1.26 Compound 1.23, wherein R³ is selected from methyl, ethyl, propyl, isopropyl, but-1-yl (n-butyl), but-2-yl (sec-butyl, e.g., (S)-but-2-yl), 2-methylprop-1-yl (isobutyl), tert-butyl, pent-1-yl (n-pentyl), pent-2-yl (e.g. (S)-pent-2-yl), 3-methyl-but-1-yl (isopentyl), 3-methylbut-2-yl, pent-3-yl, 2-methyl-but-1-yl, 2,2-dimethylpropyl (neopentyl), tert-pentyl, hex-1-yl (n-hexyl), hex-2-yl, hex-3-yl, 2-methylpent-1-yl, 4-methyl-pent-2-yl, and 3,3-dimethylbut-1-yl (neohexyl);
1.27 Compound 1.23, wherein R³ is C₁₋₆alkyl substituted with C₃₋₆cycloalkyl and optionally further substituted (e.g., R³ is optionally substituted C₁₋₃alkylC₃₋₆cycloalkyl);
1.28 Compound 1.27, wherein R³ is selected from cyclopropylmethyl, (1-methylcyclopropyl)methyl, cyclobutylmethyl, cyclopentylmethyl, 1-cyclopropylethyl, 2-cyclopropylethyl, 1-cyclobutylethyl, 2-cyclobutylethyl, 2-cyclopentylethyl, 2-cyclopentylethyl, 3-cyclopropylpropyl, cyclopent-3-en-1-ylmethyl, and cyclohexylmethyl;
1.29 Compound 1.18, wherein R³ is optionally substituted C₂₋₆alkenyl or C₂₋₆alkynyl;
1.30 Compound 1.29, wherein R³ is unsubstituted C₂₋₆alkenyl (e.g., allyl);
1.31 Compound 1.29, wherein R³ is substituted C₂₋₆alkenyl;
1.32 Compound 1.29, wherein R³ is unsubstituted C₂₋₆alkynyl;
1.33 Compound 1.29, wherein R³ is substituted C₂₋₆alkynyl;
1.34 Compound 1.29, wherein R³ is selected from but-3-en-1-yl, pent-2-en-1-yl (e.g., (E)-pent-2-en-1-yl), pent-3-en-1-yl (e.g., (E)-pent-3-en-1-yl), pent-4-en-2-yl, 2-methylbut-3-en-1-yl, 3-methylbut-2-en-1-yl, but-3-yn-1-yl, pent-4-yn-1-yl, and pent-4-yn-2-yl;
1.35 Compound 1.18, wherein R³ is optionally substituted C₃₋₆cycloalkyl or optionally substituted C₃₋₇cycloalkyl;
1.36 Compound 1.35, wherein R³ is unsubstituted C₃₋₆cycloalkyl;
1.37 Compound 1.35, wherein R³ is substituted C₃₋₆cycloalkyl (e.g., C₃₋₆cycloalkyl substituted with C₁₋₆alkyl);
1.38 Compound 1.35, wherein R³ is unsubstituted C₃₋₇cycloalkyl;
1.39 Compound 1.35, wherein R³ is substituted C₃₋₇cycloalkyl (e.g., C₃₋₇cycloalkyl substituted with C₁₋₆alkyl);
1.40 Compound 1.35, wherein R³ is selected from cyclopropyl, cyclobutyl, 1-methylcyclobutyl, cyclopentyl, 3-methylcyclopentyl, cyclohexyl, 1-methylcyclopentyl, 2-methylcyclopent-1-yl, 2-methylcyclohexyl (e.g., (1S,2S)-2-methylcyclohexyl), 3-methylcyclohexyl, 4-methylcyclohexyl (e.g., (1R,2R)-4-methylcyclohexyl or (1S,4S)-4-methylcyclohexyl), and 2,2-dimethylcyclopent-1-yl, optionally wherein R³ is cycloheptyl;
1.41 Compound 1.18, wherein R³ is optionally substituted C₃₋₇cycloalkenyl (e.g., C₃₋₇cycloalkenyl substituted with C₁₋₆alkyl);
1.42 Compound 1.40, wherein R³ is cyclopentene-4-yl, cyclohex-2-en-1-yl, or cyclohex-3-en-1-yl;
1.43 Compound 1.18, wherein R³ is an optionally substituted bridged bicyclic C₃₋₆cyclolkyl or C₃₋₇cyclolkyl;
1.44 Compound 1.43, wherein R³ is selected from bicyclo[2.2.1]hept-5-en-2-yl (e.g., (1S,4S)-bicyclo[2.2.1]hept-5-en-2-yl), pent-3-yn-1-yl, bicyclo[3.1.0]hexan-3-yl, and bicyclo[2.2.1]heptan-2-yl (e.g., (1R,2R,4S)-bicyclo[2.2.1]heptan-2-yl);
1.45 Compound 1, or any of 1.5-1.9, wherein R³ is selected from methyl, propyl, allyl, and 3-methylbut-2-en-1-yl;
1.46 Compound 1.18, wherein R³ is unsubstituted heterocycloalkyl;
1.47 Compound 1.18, wherein R³ is substituted heterocycloalkyl;
1.48 Compound 1, or any of 1.1-1.47, wherein R⁴ is unsubstituted C₁₋₆alkyl;
1.49 Compound 1, or any of 1.1-1.48, wherein the compound of Formula I is a compound of Formula Ta:

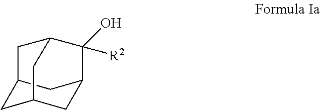

wherein R² is as defined in any preceding formula;
1.50 Compound 1.49, wherein R² is optionally substituted C₁₋₆alkyl or optionally substituted C₃₋₆cycloalkyl;
1.51 Compound 1.49, wherein R² is selected from methyl, ethyl, n-propyl, isopropyl, but-1-yl (n-butyl), but-2-yl (sec-butyl), 2-methyl-prop-1-yl (isobutyl), tert-butyl, pent-1-yl (n-pentyl), pent-2-yl (sec-pentyl), 3-methylbut-1-yl (isopentyl), pent-3-yl, hex-1-yl (n-hexyl), cyclopropyl, cyclobutyl, cyclopentyl, and cyclohexyl;
1.52 Compound 1, or any of 1.1-1.48, wherein the compound of Formula I is a compound of Formula Ib:

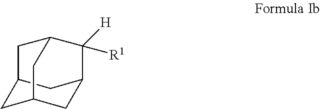

wherein R¹ is CH₂COOH or CH₂COOR³ and R³ is as defined in any preceding formula;
1.53 Compound 1.52, wherein R¹ is CH₂COOR³ and R³ is optionally substituted C₁₋₆alkyl (e.g., C₁₋₆alkyl substituted with C₁₋₆alkyl);
1.54 Compound 1.53, wherein R¹ is CH₂COOR³ and R³ is selected from methyl, ethyl, propyl, isopropyl, but-1-yl (n-butyl), but-2-yl (sec-butyl, e.g., (S)-but-2-yl), 2-methylprop-1-yl (isobutyl), tert-butyl, pent-1-yl (n-pentyl), pent-2-yl (e.g. (S)-pent-2-yl), 3-methyl-but-1-yl (isopentyl), 3-methylbut-2-yl, pent-3-yl, 2-methyl-but-1-yl, 2,2-dimethylpropyl (neopentyl), tert-pentyl, hex-I-yl (n-hexyl), hex-2-yl, hex-3-yl, 2-methylpent-1-yl, 4-methyl-pent-2-yl, and 3,3-dimethylbut-1-yl (neohexyl);
1.55 Compound 1.52, wherein R¹ is CH₂COOR³ and R³ is optionally substituted C₂₋₆alkenyl, or C₂₋₆alkynyl;
1.56 Compound 1.55, wherein R¹ is CH₂COOR³ and R³ is selected from but-3-en-1-yl, pent-2-en-1-yl (e.g., (E)-pent-2-en-1-yl), pent-3-en-1-yl (e.g., (E)-pent-3-en-1-yl), pent-4-en-2-yl, 2-methylbut-3-en-1-yl, 3-methylbut-2-en-1-yl, but-3-yn-1-yl, pent-4-yn-1-yl, and pent-4-yn-2-yl;
1.57 Compound 1.52, wherein R¹ is CH₂COOR³ and R³ is optionally substituted C₃₋₆cycloalkyl (e.g., C₃₋₆cycloalkyl substituted with C₁₋₆alkyl);
1.58 Compound 1.52, wherein R¹ is CH₂COOR³ and R³ is optionally substituted C₃₋₇cycloalkyl (e.g., C₃₋₇cycloalkyl substituted with C₁₋₆alkyl);
1.59 Compound 1.57 or 1.58, wherein R¹ is CH₂COOR³ and R³ is selected from cyclopropyl, cyclobutyl, 1-methylcyclobutyl, cyclopentyl, 3-methylcyclopentyl, cyclohexyl, 1-methylcyclopentyl, 2-methylcyclopent-1-yl, 2-methylcyclohexyl (e.g., (1S,2S)-2-methylcyclohexyl), 3-methylcyclohexyl, 4-methylcyclohexyl (e.g., (1R,2R)-4-methylcyclohexyl or (1S,4S)-4-methylcyclohexyl), and 2,2-dimethylcyclopent-1-yl, optionally wherein R³ is cycloheptyl;
1.60 Compound 1.52, wherein R¹ is CH₂COOR³ and R³ is optionally substituted C₃₋₆cycloalkenyl (e.g., C₃₋₆cycloalkenyl substituted with C₁₋₆alkyl);
1.61 Compound 1.60, wherein R¹ is CH₂COOR³ and R³ is cyclopentene-4-yl, cyclohex-2-en-1-yl, or cyclohex-3-en-1-yl;
1.62 Compound 1.52, wherein R¹ is CH₂COOR³ and R³ is C₁₋₆alkyl substituted with C₃₋₆cycloalkyl and optionally further substituted (e.g., R³ is optionally substituted C₁₋₃alkylC₃₋₆cycloalkyl);
1.63 Compound 1.62, wherein R¹ is CH₂COOR³ and R³ is selected from cyclopropylmethyl, (1-methylcyclopropyl)methyl, cyclobutylmethyl, cyclopentylmethyl, 1-cyclopropylethyl, 2-cyclopropylethyl, 1-cyclobutylethyl, 2-cyclobutylethyl, 2-cyclopentylethyl, 2-cyclopentylethyl, 3-cyclopropylpropyl, cyclopent-3-en-1-ylmethyl, and cyclohexylmethyl;
1.64 Compound 1.52, wherein R¹ is CH₂COOR³ and R³ is an optionally substituted bridged bicyclic C₃₋₆cyclolkyl or C₃₋₇cyclolkyl;
1.65 Compound 1.64, wherein R¹ is CH₂COOR³ and R³ is selected from bicyclo[2.2.1]hept-5-en-2-yl (e.g., (1S,4S)-bicyclo[2.2.1]hept-5-en-2-yl), pent-3-yn-1-yl, bicyclo[3.1.0]hexan-3-yl, and bicyclo[2.2.1]heptan-2-yl (e.g., (1R,2R,4S)-bicyclo[2.2.1]heptan-2-yl);
1.66 Compound 1, or any of 1.1-1.48, wherein the compound of Formula I is a compound of Formula Ib:

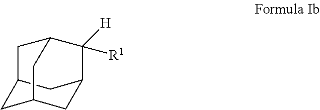

wherein R¹ is OR³ and R³ is as defined in any preceding formula;
1.67 Compound 1.66, wherein R¹ is OR³ and R³ is C₁₋₆alkyl, C₂₋₆alkenyl, or C₂₋₆ alkynyl;
1.68 Compound 1.66, wherein R¹ is OR³ and R³ is C₁₋₆alkyl or C₂₋₆alkenyl;
1.69 Compound 1.66, wherein R¹ is OR³ and R³ is selected from methyl, propyl, allyl, and 3-methylbut-2-en-1-yl;
1.70 Compound 1, or any of 1.1-1.48, wherein the compound of Formula I is:

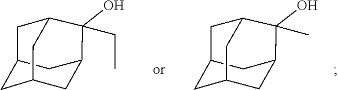

1.71 Any preceding compound, wherein the compound is selected from one or more of the compounds of Example 1-93, e.g., any of Examples 1-38, 40-66, 68-83, and 84-93; or any of Examples 1-6, 8-10, 12-20, 22-24, 26, 27, 29-31, 33-44, 46-50, 52-89, and 92; or any of Examples 1-6, 8-10, 12-20, 22-24, 26, 27, 29-31, 33-38, 40-44, 46-50, 52-66, 68-83, 85-89, and 92;
1.72 Any preceding compound, wherein the compound has a molecular weight selected from the range of to 150 to 400, or 150 to 350, or 150 to 300, or 150 to 250, or 150 to 225, or 150 to 200, or 150 to 190, or 175 to 350, or 175 to 300, or 175 to 250, or 175 to 225, or 175 to 200, or 175 to 190;
1.73 Any preceding compound wherein if the compound has an acidic or basic atom or functional group, then the compound is in the form of a salt, e.g., a base addition salt or an acid addition salt;
1.74 Any preceding compound, wherein the compound has a pleasing taste and/or aroma, e.g., as judged by a trained flavor or fragrance chemist or master perfumer (e.g., patchouli, camphoraceous, citrus and/or zesty aromas and/or tastes).

In a second aspect, the present disclosure provides a flavor composition and/or a fragrance composition (Composition 1) comprising Compound 1, or any of 1.1-1.74, in admixture with one or more non-toxic, orally acceptable, pharmaceutically acceptable, cosmetically acceptable, or acceptable for a household product, carriers or excipients. In particular embodiments, the second aspect provides:
1.1 Composition 1, wherein the composition is a fragrance composition.
1.2 Composition 1, wherein the composition is a flavor composition.
1.3 Composition 1, or any of Compositions 1.1-1.2, wherein the composition comprises the Compound 1, or any of 1.1-1.74, in an amount of 0.1 to 100% by weight of the composition, e.g., 0.1 to 90%, or 0.1 to 80%, or 0.1 to 70%, or 0.1 to 60%, or 0.1 to 50%, or 0.1 to 40%, or 0.1 to 30%, or 0.1 to 20%, or 0.1 to 15%, or 0.1 to 10%, or 0.1 to 7.5%, or 0.1 to 5%, or 0.1 to 4%, or 0.1 to 3%, or 0.1 to 2%, or 0.1 to 1%, or 10 to 100%, or 20 to 100%, or 30 to 100%, or 40 to 100%, or 50 to 100%, or 60 to 100%, or 70 to 100%, or 80 to 100%, or 90 to 100%, or 95 to 100%, or 25 to 75%, or 50 to 75%, or 75 to 95%, by weight of the composition.
1.4 Composition 1, or any of Compositions 1.1-1.3, wherein the composition further comprises one or more other flavors or fragrances.
1.5 Composition 1, or any of Compositions 1.1-1.4, wherein the composition further comprises one or more solvents.
1.6 Composition 1.5, wherein the one or more solvents are selected from water, methanol, ethanol, propanol, isopropanol, dimethyl ether, diethyl ether, diisopropyl ether, methyl tert-butyl ether, ethylene glycol, propylene glycol, glycerin, triethyl citrate, triacetin, triglycerides, liquid waxes, propylene glycol derivatives (e.g., polypropylene glycols or ethylene oxide/propylene oxide copolymers), ethylene glycol derivatives (e.g., polyethylene glycols or ethylene oxide/propylene oxide copolymers), other alcohols or ethers, or any combination thereof, optionally wherein the solvent comprises or consists of ethanol.
1.7 Composition 1, or any of composition 1.1 to 1.6, wherein the composition is a liquid.
1.8 Composition 1, or any of compositions 1.1 to 1.6, wherein the composition is a soft or waxy solid.
1.9 Composition 1, or any of compositions 1.1-1.8, wherein the composition further comprises one or more of a polymer, gelling agent, powdery substrate, surfactant, emollient, plasticizer, wetting agent, swelling agent, or active agent (e.g., an oral care active or a medicinal active agent), or any other additives as described herein.
1.10 Composition 1, or any of Compositions 1.1-1.9, wherein the composition does not comprise any ingredient or component that would not be safe for ingestion, application to the oral cavity, or topical application to the skin or hair;
1.11 Composition 1, or any of Compositions 1.1-1.10, wherein the composition does not comprise any ingredient or component which is unsafe for, or not approved for, use in a food, cosmetic composition, pharmaceutical composition, oral care composition, or consumer cleaning composition.

As used herein, the term “fragrance composition” means a mixture of fragrance ingredients (e.g., including a Compound of Formula I) with one or more non-toxic, cosmetically acceptable, or acceptable for a household product, carriers or excipients, such as solvents. For example, the fragrance ingredient(s) may be dissolved in a suitable solvent or mixed with a powdery substrate, with additional auxiliary substances added (e.g., additives), if desired. A fragrance composition is used, and intended to be used, to provide or impart a desired odor or aroma to a product, such as a cosmetic product or household product (e.g., household cleaners). Thus, a fragrance composition is used as an ingredient or component in a final product, such as a cosmetic product or consumer product, for which a particular fragrance is desired. Examples of products having fragrance compositions include, but are not limited to, perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, and pet litter. A fragrance composition should have enough of its fragrance ingredients so that it is effective to provide the desired odor or aroma to the final product, and this depends both on the concentration of the fragrance ingredients in the composition and the concentration of the composition used in the product.

As used herein, the term “flavor composition” means a mixture of flavor ingredients (e.g., including a Compound of Formula I) with one or more non-toxic, orally acceptable, or pharmaceutically acceptable, carriers or excipients, such as solvents. For example, the flavor ingredient(s) may be dissolved in a suitable solvent or mixed with a suitable solid, semi-solid, or liquid excipients, with additional auxiliary substances added (e.g., additives), if desired. A flavor composition is used, and intended to be used, to provide or impart a desired flavor and aroma to a product, such as a food product or oral pharmaceutical product. Thus, a flavor composition is used as an ingredient or component in a final product, such as a food or oral pharmaceutical product, for which a particular flavor is desired. Examples of products having flavor compositions include, but are not limited to, oral care compositions (e.g., dental hygiene products such as mouth wash, toothpaste, floss, and breath fresheners), pharmaceutical compositions (e.g., orally administered medications including liquids, tablets or capsules), and food products. A flavor composition should have enough of its flavor ingredients so that it is effective to provide the desired flavor and aroma to the final product, and this depends both on the concentration of the flavor ingredients in the composition and the concentration of the composition used in the product.

Fragrance and flavor ingredients and mixtures of fragrance and flavor ingredients that may be used in combination with the disclosed compound for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products. As used herein, the terms “fragrance ingredient” and “flavor ingredient” refer to ingredients other than the Compounds of Formula I which are used to impart a flavor or a fragrance to a composition or product.

Examples of esters which may be used as fragrance ingredients or flavor ingredients in the compositions and products of the present disclosure include, but are not limited to, acrylic acid esters (methyl, ethyl, etc.), acetoacetic acid esters (methyl, ethyl, etc.), anisic acid esters (methyl, ethyl, etc.), benzoic acid esters (allyl, isoamyl, ethyl, geranyl, linalyl, phenylethyl, hexyl, cis-3-hexenyl, benzyl, methyl, etc.), anthranilic acid esters (cinnamyl, cis-3-hexenyl, methyl, ethyl, linalyl, isobutyl, etc.), N-methylanthranilic acid esters (methyl, ethyl, etc.), isovaleric acid esters (amyl, allyl, isoamyl, isobutyl, isopropyl, ethyl, octyl, geranyl, cyclohexyl, citronellyl, terpenyl, linalyl, cinnamyl, phenylethyl, butyl, propyl, hexyl, benzyl, methyl, rhodinyl, etc.), isobutyric acid esters (isoamyl, geranyl, citronellyl, terpenyl, cinnamyl, octyl, nellyl, phenylethyl, phenylpropyl, phenoxyethyl, butyl, propyl, isopropyl, hexyl, benzyl, methyl, ethyl, linalyl, rhodinyl, etc.), undecylenic acid esters (allyl, isoamyl, butyl, ethyl, methyl, etc.), octanoic acid esters (allyl, isoamyl, ethyl, octyl, hexyl, butyl, methyl, linalyl, etc.), octenoic acid esters (methyl, ethyl, etc.), octynecarboxylic acid esters (methyl, ethyl, etc.), caproic acid esters (allyl, amyl, isoamyl, methyl, ethyl, isobutyl, propyl, hexyl, cis-3-hexenyl, trans-2-hexenyl, linalyl, geranyl, cyclohexyl, etc.), hexenoic acid esters (methyl, ethyl, etc.), valeric acid esters (amyl, isopropyl, isobutyl, ethyl, cis-3-hexenyl, trans-2-hexenyl, cinnamyl, phenylethyl, methyl, etc.), formic acid esters (anisyl, isoamyl, isopropyl, ethyl, octyl, geranyl, citronellyl, cinnamyl, cyclohexyl, terpenyl, phenylethyl, butyl, propyl, hexyl, cis-3-hexenyl, benzyl, linalyl, rhodinyl, etc.), crotonic acid esters (isobutyl, ethyl, cyclohexyl, etc.), cinnamic acid esters (allyl, ethyl, methyl, isopropyl, propyl, 3-phenylpropyl, benzyl, cyclohexyl, methyl, etc.), succinic acid esters (monomenthyl, diethyl, dimethyl, etc.), acetic acid esters (anisyl, amyl, α-amylcinnamyl, isoamyl, isobutyl, isopropyl, isobornyl, isoeugenyl, eugenyl, 2-ethylbutyl, ethyl, 3-octyl, p-cresyl, o-cresyl, geranyl, α- or β-santalyl, cyclohexyl, cycloneryl, dihydrocuminyl, dimethyl benzyl carbinyl, cinnamyl, styralyl, decyl, dodecyl, terpenyl, guainyl, neryl, nonyl, phenyl ethyl, phenylpropyl, butyl, furfuryl, propyl, hexyl, cis-3-hexenyl, trans-2-hexenyl, cis-3-nonenyl, cis-6-noneyl, cis-3-cis-6-nonadienyl, 3-methyl-2-butenyl, heptyl, benzyl, bornyl, myrcenyl, dihydromyrcenyl, myrtenyl, methyl, 2-methylbutyl, menthyl, linalyl, rhodinyl, etc.), salicylic acid esters (allyl, isoamyl, phenyl, phenylethyl, benzyl, ethyl, methyl, etc.), cyclohexylalkanoic acid esters (ethyl cyclohexylacetate, allyl cyclohexylpropionate, allyl cyclohexylbutyrate, allyl cyclohexylhexanoate, allyl cyclohexyldecanoate, allyl cyclohexylvalerate, etc.), stearic acid esters (ethyl, propyl, butyl, etc.), sebacic acid esters (diethyl, dimethyl, etc.), decanoic acid esters (isoamyl, ethyl, butyl, methyl, etc.), dodecanoic acid esters (isoamyl, ethyl, butyl, etc.), lactic acid esters (isoamyl, ethyl, butyl, etc.), nonanoic acid esters (ethyl, phenylethyl, methyl, etc.), nonenoic acid esters (allyl, ethyl, methyl, etc.), hydroxyhexanoic acid esters (ethyl, methyl, etc.), phenylacetic acid esters (isoamyl, isobutyl, ethyl, geranyl, citronellyl, cis-3-hexenyl, methyl, etc.), phenoxyacetic acid esters (allyl, ethyl, methyl, etc.), furancarboxylic acid esters (ethyl furancarboxylate, methyl furancarboxylate, hexyl furancarboxylate, isobutyl furaneopentyl glycol diacetateropionate, etc.), propionic acid esters (anisyl, allyl, ethyl, amyl, isoamyl, propyl, butyl, isobutyl, isopropyl, benzyl, geranyl, cyclohexyl, citronellyl, cinnamyl, tetrahydrofurfuryl, tricyclodecenyl, heptyl, bornyl, methyl, menthyl, linallyl, terpenyl, α-methylpropionyl, β-methylpropionyl, etc.), heptanoic acid esters (allyl, ethyl, octyl, propyl, methyl, etc.), heptenecarboxylic acid esters (allyl, ethyl, propyl, methyl, etc.), myristic acid esters (isopropyl, ethyl, methyl, etc.), phenylglycidic acid esters (ethyl phenylglycidate, ethyl 3-methylphenylglycidate, ethyl p-methyl-β-phenylglycidate, etc.), 2-methylbutyric acid esters (methyl, ethyl, octyl, phenyl ethyl, butyl, hexyl, benzyl, etc.), 3-methylbutyric acid esters (methyl, ethyl, etc.), butyric acid esters (anisyl, amyl, allyl, isoamyl, methyl, ethyl, propyl, octyl, guainyl, linallyl, geranyl, cyclohexyl, citronellyl, cinnamyl, nellyl, terpenyl, phenylpropyl, β-phenylethyl, butyl, hexyl, cis-3-hexenyl, trans-2-hexenyl, benzyl, rhodinyl, etc.), and hydroxybutyric acid esters (methyl, ethyl, menthyl or the like of 3-hydroxybutyric acid esters).

Examples of alcohols which may be used as fragrance ingredients or flavor ingredients, or as solvents, in the compositions and products of the present disclosure include, but are not limited to, aliphatic alcohols (isoamyl alcohol, 2-ethylhexanol, 1-octanol, 3-octanol, 1-octene-3-ol, 1-decanol, 1-dodecanol, 2,6-nonadienol, nonanol, 2-nonanol, cis-6-nonenol, trans-2, cis-6-nonadienol, cis-3, cis-6-nonadienol, butanol, hexanol, cis-3-hexenol, trans-2-hexenol, 1-undecanol, heptanol, 2-heptanol, 3-methyl-1-pentanol, etc.); terpene alcohols (borneol, isobomeol, carveol, geraniol, α- or β-santalol, citronellol, 4-thujanol, terpineol, 4-terpineol, nerol, myrcenol, myrtenol, dihydromyrcenol, tetrahydromyrcenol, nerolidol, hydroxycitronellol, farnesol, perilla alcohol, rhodinol, linalool, etc.); and aromatic alcohols (anisic alcohol, α-amylcinnamic alcohol, isopropylbenzylcarbinol, carvacrol, cumin alcohol, dimethylbenzylcarbinol, cinnamic alcohol, phenyl allyl alcohol, phenylethylcarbinol, β-phenylethyl alcohol, 3-phenylpropyl alcohol, benzyl alcohol, etc.).

Examples of aldehydes which may be used as fragrance ingredients or flavor ingredients in the compositions and products of the present disclosure include, but are not limited to, aliphatic aldehydes (acetaldehyde, octanal, nonanal, decanal, undecanal, 2,6-dimethyl-5-heptanal, 3,5,5-trimethylhexanal, cis-3, cis-6-nonadienal, trans-2, cis-6-nonadienal, valeraldehyde, propanal, isopropanal, hexanal, trans-2-hexenal, cis-3-hexenal, 2-pentenal, dodecanal, tetradecanal, trans-4-decenal, trans-2-tridecenal, trans-2-dodecenal, trans-2-undecenal, 2,4-hexadienal, cis-6-nonenal, trans-2-nonenal, 2-methylbutanal, etc.); aromatic aldehydes (anisic aldehyde, α-amylcinnamic aldehyde, α-methylcinnamic aldehyde, cyclamen aldehyde, p-isopropylphenylacetaldehyde, ethylvanillin, cumin aldehyde, salicylaldehyde, cinnamic aldehyde, o-, m- or p-tolylaldehyde, vanillin, piperonal, phenylacetaldehyde, heliotropin, benzaldehyde, 4-methyl-2-pheny-2-pentenal, p-methoxycinnamic aldehyde, p-methoxybenzaldehyde, etc.); and terpene aldehydes (geranial, citral, citronellal, α-sinensal, β-sinensal, perillaldehyde, hydroxycitronellal, tetrahydrocitral, myrtenal, cyclocitral, isocyclocitral, citronellyloxyacetaldehyde, neral, α-methylenecitronellal, myracaldehyde, vernaldehyde, safranal, etc.).

Examples of ketones which may be used as fragrance ingredients or flavor ingredients in the compositions and products of the present disclosure include, but are not limited to, cyclic ketones (1-acetyl-3,3-dimethyl-1-cyclohexene, cis-jasmone, α-, β- or γ-irone, ethyl maltol, cyclotene, dihydronootkatone, 3,4-dimethyl-1,2-cyclopentadione, sotolon, α-, β-, γ- or δ-damascone, α-, β- or γ-damascenone, nootkatone, 2-sec-butylcyclohexanone, maltol, α-, β- or γ-ionone, α-, β- or γ-methylionone, α-, β- or γ-isomethylionone, furaneol, camphor, etc.); aromatic ketones (acetonaphthone, acetophenone, anisylideneacetone, raspberry ketone, p-methyl acetophenone, anisylacetone, p-methoxy acetophenone, etc.); and chain ketones (diacetyl, 2-nonanone, diacetyl, 2-heptanone, 2,3-heptanedione, 2-pentanone, methyl amyl ketone, methyl nonyl ketone, β-methyl naphthyl ketone, methyl heptanone, 3-heptanone, 4-heptanone, 3-octanone, 2,3-hexanedione, 2-undecanone, dimethyloctenone, 6-methyl-5-hepten-2-one, etc.).

Examples of acetals which may be used as fragrance ingredients or flavor ingredients in the compositions and products of the present disclosure include, but are not limited to, acetaldehyde diethyl acetal, acetaldehyde diamyl acetal, acetaldehyde dihexyl acetal, acetaldehyde propylene glycol acetal, acetaldehyde ethyl cis-3-hexenyl acetal, benzaldehyde glycerin acetal, benzaldehyde propylene glycol acetal, citral dimethyl acetal, citral diethyl acetal, citral propylene glycol acetal, citral ethylene glycol acetal, phenylacetaldehyde dimethyl acetal, citronellyl methyl acetal, acetaldehyde phenylethylpropyl acetal, hexanal dimethyl acetal, hexanal dihexyl acetal, hexanal propylene glycol acetal, trans-2-hexenal diethyl acetal, trans-2-hexenal propylene glycol acetal, cis-3-hexenal diethyl acetal, heptanal diethyl acetal, heptanal ethylene glycol acetal, octanal dimethyl acetal, nonanal dimethyl acetal, decanal dimethyl acetal, decanal diethyl acetal, 2-methylundecanal dimethyl acetal, citronellal dimethyl acetal, Ambersage (manufactured by Givaudan), ethyl acetoacetate ethylene glycol acetal, and 2-phenylpropanal dimethyl acetal.

Examples of phenols which may be used as fragrance ingredients or flavor ingredients in the compositions and products of the present disclosure include, but are not limited to, eugenol, isoeugenol, 2-methoxy-4-vinylphenol, thymol, carvacrol, guaiacol, and chavicol, and vanillin.

Examples of ethers and epoxides which may be used as fragrance ingredients or flavor ingredients in the compositions and products of the present disclosure, but are not limited to, anethole, 1,4-cineole, dibenzyl ether, linalool oxide, limonene oxide, nerol oxide, rose oxide, methyl isoeugenol, methyl chavicol, isoamyl phenyl ethyl ether, β-napthyl methyl ether, phenyl propyl ether, p-cresyl methyl ether, vanillyl butyl ether, α-terpinyl methyl ether, citronellyl ethyl ether, geranyl ethyl ether, rose furan, theaspirane, decylmethyl ether, and methylphenyl methyl ether.

Examples of lactones which may be used as fragrance ingredients or flavor ingredients in the compositions and products of the present disclosure include, but are not limited to, γ- or δ-decalactone, γ-heptalactone, γ-nonalactone, γ- or δ-hexylactone, γ- or δ-octalactone, γ- or δ-undecalactone, δ-dodecalactone, δ-2-decenolactone, methyl lactone, 5-hydroxy-8-undecenoic acid δ-lactone, jasmine lactone, menthalactone, dihydrocoumarin, octahydrocoumarin, and 6-methylcoumarin.

Examples of furans which may be used as fragrance ingredients or flavor ingredients in the compositions and products of the present disclosure include, but are not limited to, furan, 2-methylfuran, 3-methylfuran, 2-ethylfuran, 2,5-diethyltetrahydrofuran, 3-hydroxy-2-methyltetrahydrofuran, 2-(methoxymethyl)furan, 2,3-dihydrofuran, furfural, 5-methylfurfural, 3-(2-furyl)-2-methyl-2-propenal, 5-(hydroxymethyl)furfural, 2,5-dimethyl-4-hydroxy-3(2H)-furanone (furaneol), 4,5-dim ethyl-3-hydroxy-2(5H)-furanone (sotolon), 2-ethyl-4-hydroxy-5-methyl-3(2H)-furanone (homofuraneol), 5-ethyl-3-hydroxy-4-methyl-2(5H)-furanone (homosotolon), 3-methyl-1,2-cyclopentanedione (cyclotene), 2(5H)-furanone, 4-methyl-2(5H)-furanone, 5-methyl-2(5H)-furanone, 2-methyl-3(2H)-furanone, 5-methyl-3(2H)-furanone, 2-acetylfuranone, 2-acetyl-5-methylfuran, furfuryl alcohol, methyl 2-furancarboxylate, ethyl 2-furancarboxylate, and furfuryl acetate.

Examples of hydrocarbons which may be used which may be used as fragrance ingredients or flavor ingredients in the compositions and products of the present disclosure include, but are not limited to, α- or β-bisabolene, β-caryophyllene, p-cymene, terpinene, terpinolene, cadinene, cedrene, longifolene, farnesene, limonene, ocimene, myrcene, α- or β-pinene, 1,3,5-undecatriene and valencene.

Examples of acids that may be used which may be used as fragrance ingredients or flavor ingredients in the compositions and products of the present disclosure include, but are not limited to, geranic acid, dodecanoic acid, myristic acid, stearic acid, lactic acid, phenylacetic acid, pyruvic acid, trans-2-methyl-2-pentenoic acid, 2-methyl-cis-3-pentenoic acid, 2-methyl-4-pentenoic acid, and cyclohexanecarboxylic acid.

The fragrance and flavor compositions of the application may comprise as additional fragrance or flavor ingredients one or more natural extracts or oils including, but not limited to, anise, orange, lemon, lime, mandarin, petitgrain, bergamot, lemon balm, grapefruit, elemi, olibanum, lemongrass, neroli, marjoram, angelica root, star anise, basil, bay, calamus, chamomile, caraway, cardamom, cassia, cinnamon, pepper, perilla, cypress, oregano, cascarilla, ginger, parsley, pine needle, sage, hyssop, tea tree, mustard, horseradish, clary sage, clove, cognac, coriander, estragon, eucalyptus, fennel, guaiac wood, dill, cajuput, wormseed, pimento, juniper, fenugreek, garlic, laurel, mace, myrrh, nutmeg, spruce, geranium, citronella, lavender, lavandin, palmarosa, rose, rosemary, sandalwood, oakmoss, cedarwood, vetiver, linaloe, bois de rose, patchouli, labdanum, cumin, thyme, ylang ylang, birch, capsicum, celery, tolu balsam, genet, immortelle, benzoin, jasmine, cassie, tuberose, reseda, marigold, mimosa, opoponax, orris, vanilla and licorice. Each of these natural extracts or oils comprises a complex mixture of chemical compounds, which may include those compounds described above. Additional fragrance ingredients may be isolated from natural products, for example, geraniol and citronellal may be isolated from citronella oil, citral may be isolated from lemon-grass oil, eugenol may be isolated from clove oil, and linalool may be isolated from rosewood oil. Animal products used in fragrance compositions include, but are not limited to, musk, ambergris, civet and castoreum. The natural ingredients described herein may also be produced synthetically, and may include the compounds disclosed herein, and be used as fragrance and/or flavor ingredients in the fragrance and flavor compositions of the present application.

Examples of fragrance ingredients used in perfumes, air fresheners, laundry detergents, pet litters, cleaning products, liquid and bar soaps, shampoos and conditioners, cosmetics, deodorants, and personal hygiene products include, but are not limited to: hexyl cinnamic aldehyde; amyl cinnamic aldehyde; amyl salicylate; hexyl salicylate; terpineol; 3,7-dimethyl-cis-2,6-octadien-1-ol; 2,6-dimethyl-2-octanol; 2,6-dimethyl-7-octen-2-ol; 3,7-dimethyl-3-octanol; 3,7-dimethyl-trans-2,6-octadien-1-ol; 3,7-dimethyl-6-octen-1-ol; 3,7-dimethyl-1-octanol; 2-methyl-3-(para-tert-butylphenyl)-propionaldehyde; 4-(4-hydroxy-4-methylpentyl)-3-cyclohexene-1-carboxaldehyde; tricyclodecenyl propionate; tricyclodecenyl acetate; anisaldehyde; 2-methyl-2-(para-iso-propylphenyl)-propionaldehyde; ethyl-3-methyl-3-phenyl glycidate; 4-(para-hydroxyphenyl)-butan-2-one; 1-(2,6,6-trimethyl-2-cyclohexen-1-yl)-2-buten-1-one; para-methoxyacetophenone; para-methoxy-alpha-phenylpropene; methyl-2-n-hexyl-3-oxo-cyclopentane carboxylate; undecalactone gamma, geraniol; geranyl acetate; linalool; linalyl acetate; tetrahydrolinalool; citronellol; citronellyl acetate; dihydromyrcenol; dihydromyrcenyl acetate; tetrahydromyrcenol; terpinyl acetate; nopol; nopyl acetate; 2-phenylethanol; 2-phenylethyl acetate; benzyl alcohol; benzyl acetate; benzyl salicylate; benzyl benzoate; styrallyl acetate; dimethylbenzylcarbinol; trichloromethylphenylcarbinyl methylphenylcarbinyl acetate; isononyl acetate; vetiveryl acetate; vetiverol; 2-methyl-3-(p-tert-butylphenyl)-propanal; 2-methyl-3-(p-isopropylphenyl)-propanal; 3-(p-tert-butylphenyl)-propanal; 4-(4-methyl-3-pentenyl)-3-cyclohexenecarbaldehyde; 4-acetoxy-3-pentyltetrahydropyran; methyl dihydrojasmonate; 2-n-heptylcyclopentanone; 3-methyl-2-pentyl-cyclopentanone; n-decanal; n-dodecanal; 9-decenol-1; phenoxyethyl isobutyrate; phenylacetaldehyde dimethylacetal; phenylacetaldehyde diethylacetal; geranonitrile; citronellonitrile; cedryl acetal; 3-isocamphylcyclohexanol; cedryl methyl ether; isolongifolanone; aubepine nitrile; aubepine; heliotropine; eugenol; vanillin; diphenyl oxide; hydroxycitronellal ionones; methyl ionones; isomethyl ionones; irones; cis-3-hexenol and esters thereof; indane musk fragrances; tetralin musk fragrances; isochroman musk fragrances; macrocyclic ketones; macrolactone musk fragrances; and ethylene brassylate.

The fragrance and flavor ingredients in a given product's fragrance or flavor composition are selected based on the intended use of the product and the product's desired aroma. For example, flavor ingredients used in toothpaste, mouth wash, and dental hygiene products may be selected to impart “freshness” and include, but are not limited to, spearmint oil, peppermint oil, star anise oil, lemon oil, and menthol.

Flavor compositions may be used to mask the unpleasant taste of orally administered medications. For example, if a medication is salty, a flavor composition that has cinnamon, raspberry, orange, maple, butterscotch, or glycyrrhiza (licorice) flavor may be used to mask the taste. If a medication is overly sweet, a flavor composition that has a berry, vanilla, or acacia flavor may render the medication more palatable. In the case of bitter tasting medications, flavor compositions that have cocoa, chocolate-mint, wild cherry, walnut, glycyrrhiza (licorice), and eriodictyon flavors might be used, whereas sour medications may be improved by flavor compositions that have fruity, citrus, or cherry flavors. These flavors may be provided by the natural or synthetic flavor ingredients discussed herein.

Examples of flavor ingredients used in flavor compositions for food products also include, but are not limited to, glucosyl steviol glycosides, isomenthols, carbonothoic acids, cassyrane, 1,5-octadien-3-ol, 2-mercaptoheptan-4-ol, 4 3-(methylthio)decanal, (4Z,7Z)-trideca-4,7-dienal, persicaria odorata oil, Amacha leaves extract, glutamyl-2-aminobutyric acid, glutamyl-2-aminobutyric acid, glutamyl-norvalyl-glycine, glutamyl-norvaline, N1-(2,3-Dimethoxybenzyl)-N2-(2-(pyridin-2-yl)ethyl) oxalamide, 1-(2-hydroxy-4-methylcyclohexyl)ethanone, Mexican lime oil, Persian lime oil, 6-methoxy-2,6-dimethylheptanal, 3,5-undecadien-2-one, 2,5-undecadien-1-ol, triethylthialdine. 4-methylpentyl 4-methylvalerate, (R)—N-(1-methoxy-4-methylpentan-2-yl)-3,4-dimethylbenzamide, 2 N-acetyl glutamate, 1,3-propanediol, Szechuan pepper extract, Tasmannia lanceolata extract, Mentha longifolia oil, mangosteen distillate, ethyl 3-(2-hydroxyphenyl)propanoate, 1-cyclopropanemethyl-4-methoxybenzene, prenyl thioisobutyrate, prenyl thioisovalerate, matairesinol, stevioside, 1-(2,4-dihydroxyphenyl)-3-(3-hydroxy-4-methoxyphenyl)propan-1-one, ethyl 5-formyloxydecanoate, 3-[3-(2-isopropyl-5-methyl-cyclohexyl)ureido]butyric acid ethyl ester, 2-Isopropyl-4-methyl-3-thiazoline, 2,6,10-trimethyl-9-undecenal, 5-mercapto-5-methyl-3-hexanone, Meyer lemon oil, teviol glycoside extract, Stevia rebaudiana, rebaudioside A 60%, rubescenamine, 4-amino-5-(3-(isopropylamino)-2,2-dimethyl-3-oxopropoxy)-2-methylquinoline-3-carboxylic acid, 3-methyl-5-(2,2,3-trimethylcyclopent-3-en-1-yl)pent-4-en-2-ol, (1-Methyl-2-(1,2,2-trimethylbicyclo[3.1.0]hex-3-ylmethyl)cyclopropyl)methanol, erospicata oil, and curly mint oil. See L. J. Marnett et al., GRAS Flavoring Substances 26, Food Technology, 44-45 (2013).

Preferred solvents and excipients for use in the compositions and products of the present disclosure include, but are not limited to, triethyl citrate, triacetin, glycerol, propylene glycol, dipropylene glycol, isopropyl myristate, ethanol, water, triglycerides, liquid waxes, propylene glycol derivatives (e.g., polymers), and ethylene glycol derivatives (e.g., polymers).

The amount of a given fragrance or flavor ingredient in a fragrance or flavor composition cannot be categorically described because it varies depending on the type product being scented or flavored, the intended use of the product, and the desired aroma and/or taste of the product. The amount of a fragrance or flavor ingredient in a fragrance or flavor composition is usually in the range of from about 1% to about 99% by mass of the fragrance composition. When the amount of the ingredient is too small, a sufficient strength of the scent or flavor may not be obtained. Further, when the amount of the ingredient is too large, a larger amount of the agent(s) needed to solubilize the ingredient may be needed, which may in turn reduce the desired aromatic or flavor properties of the end product by inhibiting volatilization or other mechanisms by which the flavor or fragrance is dispersed when the product is used or consumed. The amount of each of the fragrance and flavor ingredients in a given fragrance or flavor composition must therefore be selected based upon the aromatic and/or flavor characteristics of the selected ingredient, the overall composition of the product, and the intended aromatic and/or flavor effect.

Additives may be used in the flavor and fragrance compositions of the present disclosure. Additives that may be used include, but are not limited to, solvents, surfactants, pH adjusters, buffers, thickening agents, desiccants, emulsifiers, foaming agents, stabilizers, antioxidants, and disintegrating agents. Other fragrance and flavor composition additives will be selected in accordance with the intended use of the composition.

Solvents, for example water-soluble organic solvents, which may be used in the flavor and fragrance compositions of the present disclosure include, but are not limited to, ethanol, propanol, isopropanol, butanol, 3-methoxy-3-methyl-1-butanol, benzyl alcohol, ethyl carbitol (diethylene glycol monoethyl ether), ethylene glycol, propylene glycol, dipropylene glycol, butylene glycol, hexylene glycol, glycerin, ethylene glycol monomethyl ether, propylene glycol monomethyl ether, propylene glycol monoethyl ether, and dipropylene glycol monomethyl ether. These water-soluble solvents may be used solely or in combination. The content of the water-soluble organic solvent in the compositions of the application may be determined according to the desired composition properties, and is usually from about 1% to about 99% by mass.

Oil-soluble organic solvents which may be used with the flavor and fragrance compositions of the application include, but are not limited to, isoparaffin, paraffin, limonene, pinene, triethyl citrate, benzyl benzoate, isopropyl myristate, triacetin, and silicone.

Preferred solvents include, but are not limited to, triethyl citrate, triacetin, glycerol, ethanol, water, triglycerides, liquid waxes, propylene glycol derivatives, and ethylene glycol derivatives.

In some embodiments, the flavor and fragrance compositions and products of the present disclosure may further comprise other substances, including, but not limited to, sequestering agents, preservatives, antioxidants, deodorizers, sterilization agents, ultraviolet absorbers, pH adjusters, insecticidal components, components for protection from insects, insect repellents, colorants, excipients, and buffers. The substances used in, or in addition to, the fragrance and flavor compositions of the present application may be determined by the product in which the composition is included. When the substance is used in a flavor or fragrance composition, it may be an additive. When the substance is used alongside a flavor or fragrance composition, it may be considered as part of a product composition that comprises a fragrance or flavor composition.

Excipients that may be used in the fragrance and flavoring compositions and products of the present disclosure may vary depending on the use of the intended product and its overall composition. In some instances, the excipient may be included in the fragrance or flavor composition or may, alternatively, be independent of the composition. Excipients used in or with flavoring compositions of an orally administered medication may include, but are not limited to, tablet coatings, such as a cellulose ether hydroxypropyl methylcellulose, synthetic polymer, shellac, corn protein zein or other polysaccharides, and gelatin. In contrast, cosmetic excipients may include, but are not limited to, Carbopol 940 ETD, triethanolamine, purified water, glycerin, imidazolidinyl urea. EDTA, polyvinyl alcohol, methyl parabens phenoxyethanol 0, ethyl alcohol 1, peg 7 glyceryl cocoate, peg 6 triglyceryl caproic glycerides, acemulogar LAM V, isopropyl myristate, tegosoft CT, xanthan gum, sepicide CL, polyquaternium 7, and Vaseline oils, Additional suitable excipients for use with or in a flavor and/or fragrance composition for a given product will be readily selected by those having ordinary skill in the art.

Buffers that may be used with the fragrance and flavoring compositions of the present application may vary depending on the use of the intended product and its overall composition. In some instances, the buffer may be included in the fragrance or flavor composition or may, alternatively, be independent of the composition. Examples of buffers that may be used in or with the fragrance and flavor compositions of the application include, but are not limited to, citrates, acetates, and phosphates. For example, trisodium citrate may be used as a flavor or as a preservative, and is known to impart tartness to a flavor, but also acts as a buffer. Trisodium citrate is an ingredient in a variety of sodas and other beverages, as well as drink mixes and bratwurst. In cosmetic products, disodium hydrogen phosphate, potassium dihydrogen phosphate, disodium hydrogen phosphate and, and citric acid may be used to buffer the pH of the product. In toothpaste, calcium carbonate and/or dicalcium phosphate may be used as pH buffers. Additional suitable buffers for use with or in a flavor and/or fragrance composition for a given product will be readily selected by those having ordinary skill in the art.

In a fourth aspect, the present disclosure provides a product which comprises Composition 1 or any of 1.1 to 1.11. In some embodiments, the product may be selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.

Having now described some embodiments of the application, it should be apparent to those skilled in the art that the foregoing is merely illustrative and not limiting, having been presented by way of example only. The embodiments of the application can therefore be in other specific forms without departing from the spirit or essential characteristics thereof.

Those skilled in the art should recognize or be able to ascertain, using no more than routine experimentation, equivalents to the specific embodiments of the application. It is therefore to be understood that the embodiments described herein are presented by way of example only and that the scope of the application is thus indicated by the appended claims and equivalents thereto, and that the application may be practiced otherwise than as specifically described in the foregoing description.

The term “about,” when used to describe one of the compositions of the application, refers to a recited percentage ±5%, ±4%, 3%, ±2.5%, ±2%, +1.5%, ±1%, ±0.75%, ±0.5%, ±0.25%, or ±0.1%. In one embodiment, the term “about,” refers to a recited percentage ±5%. For example, “about 50%” refers to the range 45% to 55%. In one embodiment, the term “about,” refers to a recited percentage ±2.5%. In one embodiment, the term “about,” refers to a recited percentage ±1%. In one embodiment, the term “about,” refers to a recited percentage ±0.5%. In one embodiment, the term “about,” refers to a recited percentage ±0.1%.

As used herein, the singular forms “a,” “an,” and “the” include plural referents unless the context clearly dictates otherwise. Thus, for example, reference to “a fragrance ingredient” includes not only a single fragrance ingredient but also a combination or mixture of two or more different fragrance ingredients, reference to “an additive” includes a single additive as well as two or more additives, and the like.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, “optionally substituted” means that the indicated core or functional group is either unsubstituted or substituted by one or more groups up to the maximum permitted by the rules of valency, wherein said groups are selected from: halo, hydroxy, cyano, C₁₋₆alkyl, C₂₋₆alkenyl, C₂₋₆alkynyl, C₃₋₆cycloalkyl, C₁₋₆haloalkyl, C₁₋₆-alkoxy, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; wherein each of said C₁₋₆alkyl, C₃₋₆cycloalkyl, heterocycloalkyl, aryl or heteroaryl is further optionally substituted by one or more halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), heterocycloalkyl, aryl, and heteroaryl; and wherein each R^{x} is independently selected from hydrogen, C₁₋₆alkyl, C₂₋₆alkenyl, C₂₋₆alkynyl, C₃₋₆cycloalkyl, heterocycloalkyl, aryl and heteroaryl.

As used herein, the term “C₁₋₆-alkyl” means a saturated linear or branched free radical consisting essentially of 1 to 6 carbon atoms and a corresponding number of hydrogen atoms. Exemplary C₁₋₆-alkyl groups include methyl, ethyl, n-propyl, isopropyl, n-butyl, isobutyl, sec-butyl, tert-butyl, n-pentyl, isopentyl, neopentyl, n-hexyl, and thexyl. Other C₁₋₆-alkyl groups will be readily apparent to those of skill in the art given the benefit of the present disclosure. The terms “C₁₋₃-alkyl”, “C₁₋₄-alkyl”, etc., have equivalent meanings, i.e., saturated linear or branched free radical consisting essentially of 1 to 3 (or 4) carbon atoms and a corresponding number of hydrogen atoms. The similar terms “C₂₋₆-alkenyl,” “C₂₋₆-alkynyl,” “C₃₋₆-cycloalkyl,” “C₁₋₆-haloalkyl,” “C₁₋₆-alkoxy,” and the like, refer to corresponding functional groups having the stated number of carbon atoms, wherein “alkenyl” refers to an unsaturated linear or branched free radical having at least one double bond, “alkynyl” refers to an unsaturated linear or branched free radical having at least one triple bond, “haloalkyl” refers to an alkyl radical having at least one halogen atom attached to a carbon atom, and “alkoxy” refers to an alkyl radical having at least one oxygen atom attached to the alkyl radical and wherein the attachment point of the functional group is through the oxygen (i.e., to form an ether). Exemplary alkenyl groups include vinyl and allyl. Exemplary alkynyl groups include ethynyl and propynyl. Exemplary haloalkyl groups include fluoromethyl, difluoromethyl, trifluoromethyl, 3,3,3-trifluorethyl, and like groups with chlorine, bromine or iodine. “Cycloalkyl” refers to a carbocyclic ring attached via a ring carbon atom. Exemplary cycloalkyl groups include cyclopropyl, cyclobutyl, cyclopentyl and cyclohexyl.

The terms “C₃₋₆-cycloalkyl” and “C₃₋₇-cycloalkyl” also include bicyclic fused, bicyclic spiro-joined, polycyclic bridged, and polycyclic fused ring systems, provided that no ring is aromatic and no ring includes ant heteroatoms (e.g., only C and H atoms).

As used herein, the term “heteroaryl” means an aromatic free radical having 5 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen. Heteroaryl rings include monocyclic, bicyclic fused, and polycyclic fused ring systems provided that at least one ring of the ring system has at least one heteroatom (N, S, or O), and all rings are aromatic. Exemplary 5-membered heteroaryl groups include furyl, thienyl (thiophenyl), pyrrolyl, oxazolyl, thiazolyl, pyrazolyl, isothiazolyl, isoxazolyl, imidazolyl, triazolyl, oxadiazolyl, thiadiazolyl, and tetrazolyl. Exemplary 6-membered heteroaryl groups include pyridyl, pyrimidyl, pyrazinyl, pyridazinyl, and 1,2,4-triazinyl. Exemplary fused heteroaryl groups include benzoxazolyl, benzothiazolyl, benzisothiazolyl, benzisoxazolyl, benzimidazolyl, indolyl, quinolinyl, isoquinolinyl, quinazolinyl, and quinoxalinyl. Other heteroaryl groups will be readily apparent to those of skill in the art given the benefit of the present disclosure. In general, the heteroaryl group typically is attached to the main structure via a carbon atom. However, those of skill in the art will realize that certain other atoms, e.g., hetero ring atoms, can be attached to the main structure.

As used herein, the term “aryl” means an aromatic free radical having 5 or 6 atoms (i.e., ring atoms) that form a ring, wherein all of the ring atoms are carbon. Exemplary aryl groups include phenyl and naphthyl.

As used herein, the term “heterocycloalkyl” means an aromatic free radical having 3 to 20 atoms (i.e., ring atoms) that form a ring, wherein at least one atom (e.g., 1 to 5) of the ring atoms are carbon and at least one atom of the remaining ring atoms is a nitrogen, sulfur, or oxygen, and wherein at least one ring is non-aromatic. Heterocycloalkyl rings include monocyclic, bicyclic fused, bicyclic spiro-joined, polycyclic bridged, and polycyclic fused ring systems, provided that at least one ring of the ring system has at least one heteroatom (N, S, or O) and at least one ring of the ring system is non-aromatic (e.g., saturated). Exemplary saturated heterocycloalkyl groups include azetidinyl, aziridinyl, oxiranyl, oxetanyl, tetrahydrofuranyl, pyrrolidinyl, piperidinyl, piperazinyl, morpholinyl, and thiomorpholinyl. Heterocycloalkyl rings systems include ring systems in which an aromatic ring is fused to a nonaromatic ring, such as will be obtained by partial reduction of a polycyclic aromatic ring system. Exemplary ring systems of this category include indolinyl, tetrahydroquinolinyl, and tetrahydroisoquinolinyl. Other heterocycloalkyl groups will be readily apparent to those of skill in the art given the benefit of the present disclosure. A heterocycloalkyl groups can be attached to the main structure either through a carbon atom or a nitrogen atom of the ring.

The term “compound,” as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. The term also refers to any specific chemical compound in which one or more atoms have been replaced with one or more different isotopes of the same element.

The entire disclosure of each of the patent documents and scientific articles referred to herein is incorporated by reference in its entirety for all purposes. The function and advantages of these and other embodiments will be more fully understood from the following non-limiting examples. The examples are intended to be illustrative in nature and are not to be considered as limiting the scope of the embodiments discussed herein.

### Examples

Various compounds having a substituted adamantane core are commercially available for use as synthetic intermediates. These include the following compounds:

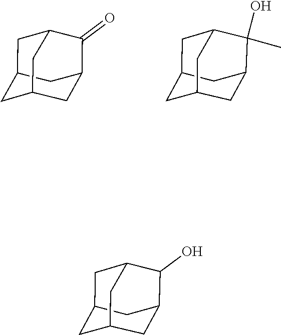

Compounds of the present disclosure can be made according to known methods published in the art. For example, the following synthetic schemes may be employed to arrive at compounds of the present disclosure:

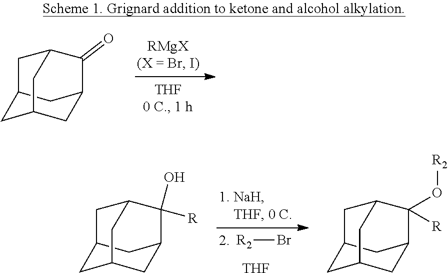

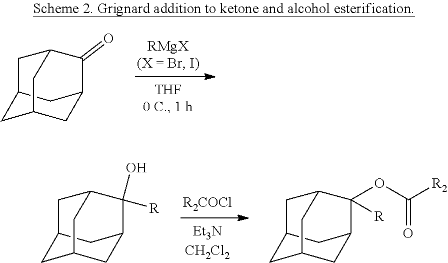

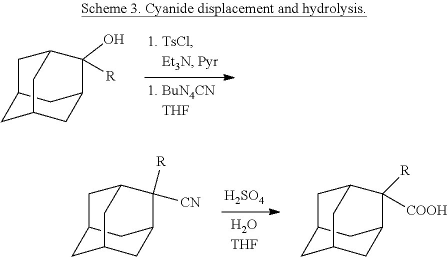

Following formation of an adamantane carboxylic acid as shown in Scheme 3, numerous other standard conversions could be carried out. For example, formation of esters or amides from the carboxylic acid (e.g., by conversion to an acid chloride followed by reaction with ana alcohol or an amine), reduction of the carboxylic acid to a hydroxymethyl group (e.g., using lithium triethylborohydride), esterification or etherification of the resulting hydroxymethyl compound according to the second steps of Scheme 1 or 2, oxidation of the resulting hydroxymethyl to an aldehyde (e.g., using Dess-Martin periodinane), and Grignard addition to the aldehyde to afford secondary alcohols, ethers and esters, according to the Scheme 1 and 2.

Compounds according to the present disclosure wherein R¹ is OH can be made according to Scheme 4.

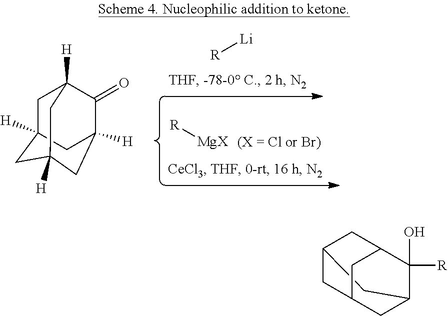

Compounds according to the present disclosure wherein R¹ is an acetic ester moiety R² is H and can be made according to Scheme 5.

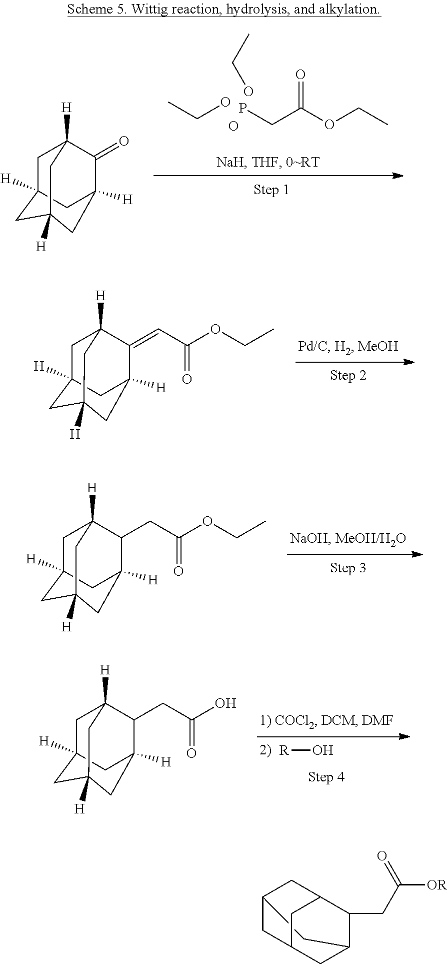

Compounds according to the present disclosure wherein R¹ is an ether and R² is H and can be made according to Scheme 6.

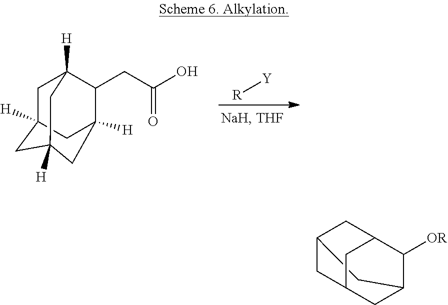

The following synthetic Examples provide exemplary embodiments within the scope of the present disclosure.

### Example 1. (1R,3S,5r,7r)-2-(sec-butyl)adamantan-2-ol

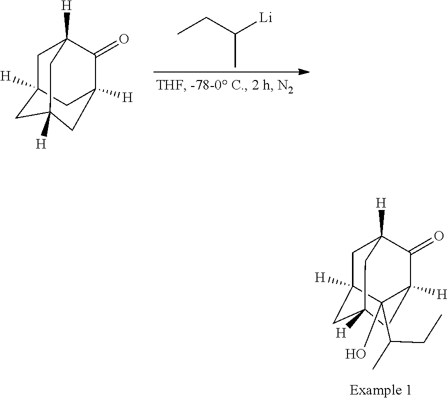

Into a 250 mL 3-round-bottom flask is added adamantan-2-one (500 mg, 3.33 mmol, 1.00 equiv.) in dry THF (100 mL) at −78° C. under N₂ atmosphere. To the mixture is added sec-butyllithium (3.84 mL, 5.00 mmol, 1.50 equiv.) dropwise over 25 min. The reaction mixture is stirred and slowly waned to 0° C. with monitoring by GC-MS. After 2 h, GC-MS indicates that the ketone is fully consumed. The reaction is quenched with water/ice (100 mL) and aqueous NaHCO₃ (3 M, 20 mL) at 0° C., and the resulting biphasic mixture is extracted with Et₂O (3×50 mL). The combined organic layers are washed with H₂O (2×20 mL), dried over anhydrous Na₂SO₄, and evaporated to afford the crude product as a white solid. The residue is purified by flash chromatography (silica gel, gradient 0% to 5% EtOAc in PE over 15 min), to afford (1R,3S,5r,7r)-2-(sec-butyl) adamantan-2-ol (416.1 mg, 60.0%) as a white solid. GC-MS: (EI+, m/z, M⁺): 208.18. ¹H-NMR: (300 MHz, DMSO-d₆, ppm) 63.56 (s, 1H)) 2.21-2.05 (m, 2H), 1.89-1.62 (m, 7H), 1.62-1.51 (m, 4H), 1.51-1.29 (i, 3H), 1.00-0.91 (i, 1H), 0.84-0.79 m, 3H), 0.78-0.69 (in, 3H).

### Examples 2 to 5. The Compounds of Examples 2 to 5 are Prepared According the Procedure of Example 1, Substituting the Appropriate Alkyl-Lithium Reagent for the Sec-Butyllithium

| | | | GC-MS | |
|---|---|---|---|---|
| Ex. | | | (EI+, | |
| No. | Structure | Compound Name | m/z, M⁺) | ¹H-NMR |
| Ex. 2 | 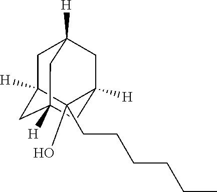 [SMILES: CCCCCCC1(O)[C@H]2C[C@@H]3C[C@@H](C[C@H]1C3)C2] | (1R,3S,5r,7r)-2- hexyladamantan-2-ol | 236.21 | ¹H-NMR (300 MHz, DMSO-d₆) δ 3.81 (s, 1H), 2.21 (d, J = 11.8 Hz, 2H), 1.74 (q, J = 9.7, 6.9 Hz, 4H), 1.65-1.47 (m, 8H), 1.44 − 1.16 (m, 11H), 0.96 − 0.76 (m, 3H). |
| Ex. 3 | 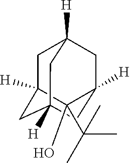 | (1R,3S,5r,7r)-2-(tert- butyl)adamantan-2-ol | 208.18 | ¹H-NMR (300 MHz, DMSO-d₆) δ 3.67 (s, 1H), 2.42 − 2.31 (m, 2H), 2.19 − 2.08 (m, 2H), 1.93 (s, 2H), 1.78 (s, 1H), 1.70 − 1.59 (m, 5H), 1.39 − 1.28 (m, 2H), 1.03 (s, 9H). |
| Ex. 4 | 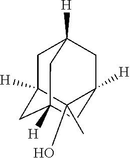 | (1R,3S,5r,7r)-2- methyladamantan-2- ol | 166.14 | ¹H-NMR (300 MHz, DMSO-d₆) δ 4.05 (s, 1H), 2.21 (d, J = 11.8 Hz, 2H), 1.86 − 1.45 (m, 10H), 1.43 − 1.28 (m, 2H), 1.20 (s, 3H). |
| Ex. 5 | 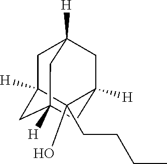 | (1R,3S,5r,7r)-2- butyladamantan-2-ol | 208.18 | ¹H-NMR (300 MHz, DMSO-d₆) δ 3.79 (s, 1H), 2.19 (d, J = 11.6 Hz, 2H), 1.80 − 1.43 (m, 12H), 1.42 − 1.11 (m, 6H), 0.98 − 0.74 (m, 3H). |

### Example 6. (1R,3S,5r,7r)-2-pentyladamnantan-2-ol

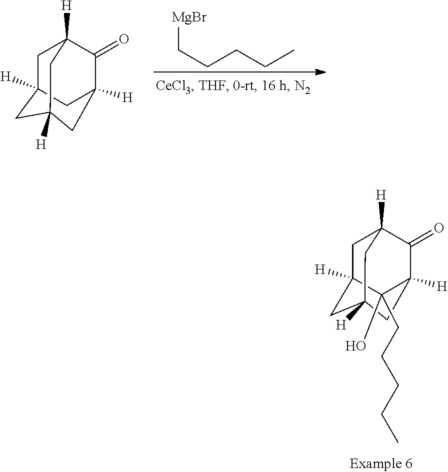

Into a 250-mL round-bottom flask is added adamantan-2-one (500 mg, 3.33 mmol, 1.00 equiv.) in dry THF (100 mL) and dry CeCl₃ (2.44 g, 10.00 mmol, 3.00 equiv.) under N₂ atmosphere at 23° C. The reaction mixture is stirred for 2 h and then immersed in an ice-water bath. n-pentylmagnesium bromide (2.0 M in THF, 5 mL, 5.00 mmol, 1.50 equiv.) is then added dropwise over 25 min while maintaining internal temperature below 5° C. The reaction mixture is stirred allowed to warm to room temperature. After 14 h, GC-MS indicated full consumption of the ketone. The reaction is quenched with water/ice (100 mL) and NaHCO₃ (3M, 20 mL) at 0° C., and the resulting mixture is extracted with Et₂O (3×50 mL). The combined organic layers are washed with H₂O (2×20 mL), dried over anhydrous Na₂SO₄, and evaporated to afford the crude product as a white solid. The residue is purified by flash chromatography (silica gel, gradient 0% to 5% EtOAc in PE over 15 min) to afford (1R,3S,5r,7r)-2-pentyladamantan-2-ol (350.00 mg, 47.3%) as a white solid. GC-MS (EI+, m/z, M⁺): 222.20. ¹H-NMR: (300 MHz, DMSO-d₆, ppm) δ 3.81 (s, 1H), 2.20 (d, J=12.1 Hz, 2H), 1.76-1.61 (m, 4H), 1.60-1.57 (m, 8H), 1.51-1.22 (m, 8H), 0.92-0.82 (m, 3H).

### Examples 7 to 19. The Compounds of Examples 7 to 19 are Prepared According the Procedure of Example 6, Substituting the Appropriate Grignard Reagent for the n-Pentylmagnesium Bromide

| | | | GCMS | |
|---|---|---|---|---|
| Ex. | | | (EI+, m/z, | |
| No. | Structure | Compound Name | M+) | ¹H-NMR |
| Ex. 7 | 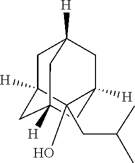 | (1R,3S,5r,7r)-2- isobutyladamantan-2-ol | 208.18 | ¹H-NMR (300 MHz, DMSO-d₆) δ 3.77 (s, 1H), 2.24 (d, J = 12.1 Hz, 2H), 1.90 − 1.68 (m, 5H), 1.62 (s, 6H), 1.49 (d, J = 5.7 Hz, 2H), 1.44 − 1.32 (m, 2H), 0.92 (d, J = 6.7 Hz, 6H). |
| Ex. 8 | 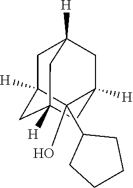 [SMILES: OC1(C2CCCC2)[C@H]2C[C@@H]3C[C@@H](C[C@H]1C3)C2] | (1R,3S,5r,7r)-2- cyclopentyladamantan-2-ol | 220.18 | ¹H-NMR (300 MHz, DMSO-d₆) δ 3.67 (s, 1H), 2.21 (d, J = 12.0 Hz, 2H), 1.85 (d, J = 12.5 Hz, 2H), 1.73 (d, J = 13.5 Hz, 2H), 1.65- 1.57 (m, 6H), 1.54-1.29 (m 10H). |
| Ex. 9 | 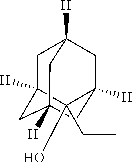 | (1R,3S,5r,7r)-2- ethyladamantan-2-ol | 180.15 | ¹H-NMR (300 MHz, DMSO-d₆) δ 3.78 (s, 1H), 2.21 (d, J = 12.0 Hz, 2H), 1.84 − 1.69 (m, 4H), 1.67 − 1.49 (m, 8H), 1.39 (d, J = 11.7 Hz, 2H), 0.80 (t, J = 7.4 Hz, 3H). |
| Ex. 10 | 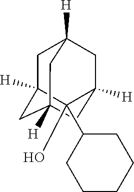 [SMILES: OC1(C2CCCCC2)[C@H]2C[C@@H]3C[C@@H](C[C@H]1C3)C2] | (1R,3S,5r,7r)-2- cyclohexyladamantan-2-ol | 234.20 | ¹H-NMR (300 MHz, DMSO-d₆) δ 3.58 (s, 1H), 2.16 (d, J = 12.1 Hz, 2H), 1.89 − 1.65 (m, 9H), 1.66 − 1.47 (m, 7H), 1.40 (d, J = 11.9 Hz, 2H), 1.28 − 0.93 (m, 5H). |
| Ex. 11 | 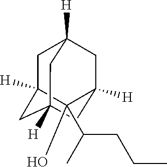 [SMILES: CCCC(C)C1(O)[C@H]2C[C@@H]3C[C@@H](C[C@H]1C3)C2] | (1R,3S,5r,7r)-2-(pentan-2- yl)adamantan-2-ol | 222.20 | ¹H-NMR (300 MHz, DMSO-d₆) δ 3.58 (s, 1H), 2.18 (d, J = 12.2 Hz, 2H), 2.01 − 1.89 (m, 1H), 1.86 (s, 1H), 1.75 (t, J = 11.4 Hz, 5H), 1.59 (d, J = 18.4 Hz, 3H), 1.46 − 1.27 (m, 4H), 1.27 − 1.08 (m, 1H), 1.08 − 0.92 (m, 1H), 0.87 (t, J = 7.1 Hz, 3H), 0.76 (d, J = |
| | | | | 6.7 Hz, 3H). |
| Ex. 12 | 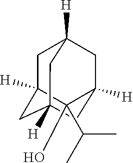 | (1R,3S,5r,7r)-2- isopropyladamantan-2-ol | 194.17 | ¹H-NMR (300 MHz, DMSO-d₆) δ 3.59 (s, 1H), 2.25-2.08 (m, 3H), 1.73 (d, J = 16.6 Hz, 6H), 1.59 (d, J = 17.1 Hz, 4H), 1.40 (d, J = 11.7 Hz, 2H), 0.77 (d, J = 6.7 Hz, 6H). |
| Ex. 13 | 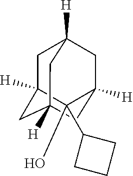 [SMILES: OC1(C2CCC2)[C@H]2C[C@@H]3C[C@@H](C[C@H]1C3)C2] | (1R,3S,5r,7r)-2- cyclobutyladamantan-2-ol | 206.17 | ¹H-NMR (300 MHz, DMSO-d₆) δ 3.84 (s, 1H), 3.04 (p, J = 8.4 Hz, 1H), 2.19 (d, J = 11.9 Hz, 2H), 2.08 − 1.85 (m, 2H), 1.87 − 1.67 (m, 5H), 1.60 (td, J = 14.4, 13.0, 9.5 Hz, 7H), 1.50 − 1.28 (m, 4H). |
| Ex. 14 | 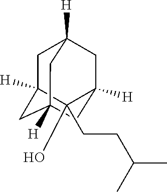 [SMILES: CC(C)CCC1(O)[C@H]2C[C@@H]3C[C@@H](C[C@H]1C3)C2] | (1R,3S,5r,7r)-2- isopentyladamantan-2-ol | 222.20 | ¹H-NMR (300 MHz, DMSO-d₆) δ 3.80 (s, 1H), 2.21 (d, J = 12.0 Hz, 2H), 1.89 − 1.65 (m, 4H), 1.67 − 1.45 (m, 8H), 1.48 − 1.26 (m, 3H), 1.25 − 0.99 (m, 2H), 0.87 (d, J = 6.5 Hz, 6H). |
| Ex. 15 | 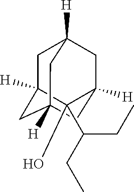 [SMILES: CCC(CC)C1(O)[C@H]2C[C@@H]3C[C@@H](C[C@H]1C3)C2] | (1R,3S,5r,7r)-2-(pentan-3- yl)ladamantan-2-ol | 222.20 | ¹H-NMR (300 MHz, DMSO-d₆) δ 3.55 (s, 1H), 2.19 (d, J = 12.1 Hz, 2H), 1.87 − 1.33 (m, 15H), 1.23-1.05 m, 2H), 0.90 (t, J = 7.5 Hz, 6H). |
| Ex. 16 | 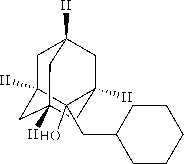 [SMILES: OC1(CC2CCCCC2)[C@H]2C[C@@H]3C[C@@H](C[C@H]1C3)C2] | (1R,3S,5r,7r)-2- (cyclohexylmethyl)adamantan- 2-ol | 248.21 | ¹H-NMR (300 MHz, DMSO-d₆) δ 3.76 (s, 1H), 2.23 (d, J = 12.1 Hz, 2H), 1.86-1.67 (m, 7H), 1.66-1.51 (m, 11H), 1.49 − 1.30 (m, 5H), 1.27-1.07 (m, 4H), 1.07- 0.87 (m, 3H). |
| Ex. 17 | 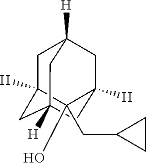 [SMILES: OC1(CC2CC2)[C@H]2C[C@@H]3C[C@@H](C[C@H]1C3)C2] | (1R,3S,5r,7r)-2- (cyclopentylmethyl) adamantan- 2-ol | 206.17 | ¹H-NMR (300 MHz, DMSO-d₆) δ 5.89 − 5.65 (m, 1H), 5.13 − 4.77 (m, 2H), 3.94 (s, 1H), 2.21 (d, J = 12.1 Hz, 2H), 2.10 − 1.95 (m, 2H), 1.86 − 1.69 (m, 4H), 1.63- 1.55 (m, 7H), 1.48 − 1.24 (m, 3H). |
| Ex. 18 | 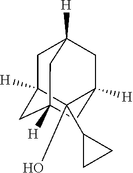 [SMILES: OC1(C2CC2)[C@H]2C[C@@H]3C[C@@H](C[C@H]1C3)C2] | (1R,3S,5r,7r)-2- cyclopentyladamantan-2-ol | 192.15 | ¹H-NMR (300 MHz, DMSO-d₆) δ 3.49 (s, 1H), 2.23 − 2.12 (m, 2H), 2.08 − 1.97 (m, 2H), 1.80 (t, J = 3.2 Hz, 1H), 1.75 − 1.59 (m, 5H), 1.56 (d, J = 3.8 Hz, 2H), 1.47 − 1.32 (m, 4H), 0.39 − 0.28 (m, 2H), 0.22 − 0.09 (m, 2H). |
| Ex. 19 | 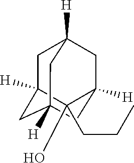 | (1R,3S,5r,7r)-2- pentyladamantan-2-ol | 194.17 | ¹H-NMR (300 MHz, DMSO-d₆) δ 3.82 (s, 1H), 2.21 (d, J = 12.0 Hz, 2H), 1.85 − 1.62 (m, 4H), 1.62 (s, 3H), 1.59 − 1.47 (m, 5H), 1.43 − 1.22 (m, 4H), 1.37 − 1.21 (m, 3H), 0.87 (t, J = 7.2 Hz, 3H) |

### Example 20. 2,2-dimethylcyclopentyl 2-((1R,3S,5r,7r)-adamantan-2-yl) acetate

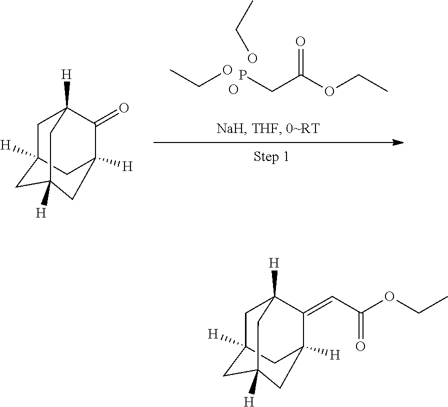

Step 1. A mixture of adamantan-2-one (100 g, 666.67 mmol, 1.00 equiv.) and NaH (20.79 g, 866.67 mmol, 1.30 equiv.) in THF (100 mL) is stirred at 0° C. for 30 min. Triethyl phosphonoacetate (179.20 g, 800.00 mmol, 1.20 equiv.) is then added and the resulting mixture is stirred and allowed to warm to 23° C. After 2 h, the reaction is quenched by the addition of sat. aq. NH₄Cl (500 mL). The resulting biphasic mixture is extracted with EtOAc (3×500 mL), and the combined organic layers are concentrated under reduced pressure. The resulting residue is purified by flash column chromatography (silica gel, PE/THF 3:1) to afford ethyl 2-[(1r,2Z,3r,5r,7r)-adamantan-2-ylidene]acetate (146 g, 99.9%) as a white solid. LC-MS (ESI+, n/z, [M+H]⁺): 221.15.

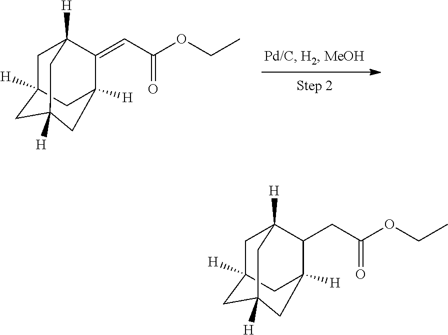 [SMILES: CCOC(=O)C=C1[C@H]2C[C@@H]3C[C@@H](C[C@H]1C3)C2 ; CCOC(=O)CC1[C@H]2C[C@@H]3C[C@@H](C[C@H]1C3)C2]

Step 2. To a solution of ethyl 2-[(1r,2Z,3r,5r,7r)-adamantan-2-ylidene]acetate (146 g, 663.63 mmol, 1.00 equiv.) in MeOH (100 mL) in a stainless-steel autoclave is added Pd/C (10%, 14.6 g). Hydrogen is added to the autoclave to a pressure of 3 psi, and the resulting mixture is stirred at 23° C. After 6 h, the pressure is released and the reaction mixture is filtered through a pad of celite, then concentrated under reduced pressure to afford ethyl 2-((1r,3r,5r,7r)-adamantan-2-yl)acetate (129 g, 89.2%) as a white solid, which was carried forward without further purification. GC-MS: (EI+, m/z, M⁺): 222.16.

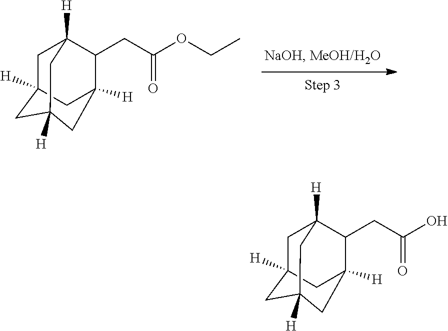 [SMILES: CCOC(=O)CC1[C@H]2C[C@@H]3C[C@@H](C[C@H]1C3)C2 ; O=C(O)CC1[C@H]2C[C@@H]3C[C@@H](C[C@H]1C3)C2]

Step 3. A solution of ethyl 2-((1r,3r,5r,7r)-adamantan-2-yl)acetate (129 g, 581.08 mmol, 1.00 equiv.) in MeOH (500 mL) and water (500 mL) is stirred at 0° C. under nitrogen atmosphere. After 5 min, NaOH (45.32 g, 1162.16 mmol, 2.00 equiv.) is added in 5 portions over 10 min. The resulting mixture is stirred and allowed to warm to 23° C. After 3 h, the mixture is neutralized to a pH of about 6 with citric acid. The reaction mixture is then filtered through a pad of celite and concentrated under reduced pressure to afford (1r,3r,5r,7r)-adamantan-2-ylacetic acid (100 g, 89.3%) as a white solid, which was carried forward without further purification. LC-MS: (ES−, m/z) [M−H]⁻): 193.

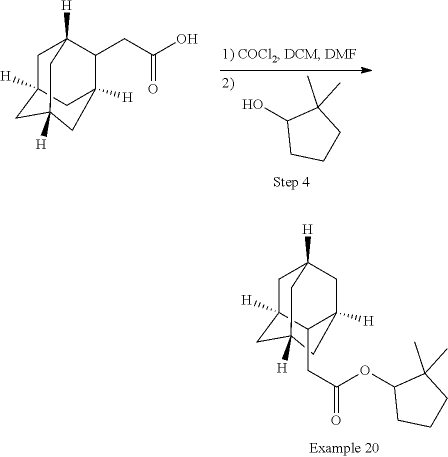

Step 4. To a 250-mL round-bottom flask is added (1r,3r,5r,7r)-adamantan-2-ylacetic acid (800 mg, 4.12 mmol, 1.00 equiv.) and dry DCM (100 mL) at 0° C. DMF (30 mg, 0.412 mmol, 0.1 equiv.) and COCl₂ (606.18 mg, 6.18 mmol, 1.50 equiv.) are then added dropwise. The reaction mixture is stirred for 1 h at 0° C. and then 2,2-dimethylcyclopentan-1-ol (7.4.52 mg, 6.18 mmol, 1.50 equiv.) is added. The resulting mixture is allowed to warm to room temperature. After 1 h, GC-MS indicates full consumption of starting material. The reaction is quenched with water/ice (100 mL). The resulting mixture is extracted Et₂O (3×50 mL) and the combined organic layers are washed with water (2×20 mL), dried over anhydrous Na₂SO₄, filtered, and concentrated under reduced pressure to afford the crude product as a wet solid. The residue is purified by flash chromatography (silica gel, gradient 0% to 5% EtOAc in PE over 15 min) to afford 2,2-dimethylcyclopentyl 2-((1R,3S,5r,7r)-adamantan-2-yl) acetate (818.1 mg, 68.4%) as a colorless oil. GC-MS: (EI+, m/z, M⁺): 290.22. ¹H-NMR: (300 MHz, DMSO-d₆, ppm) δ 4.67-4.57 (m, 1H), 2.44-2.40 (m, 2H), 2.19-1.96 (m, 2H), 1.89-1.35 (m, 19H), 0.91 (s, 6H).

### Examples 21 to 88. The Compounds of Examples 7 to 19 are Prepared According the Procedure of Example 1, Substituting the Appropriate Alcohol for the 2,2-Dimethylcyclopentan-1-Ol in Step 4

| | | | GC-MS | |
|---|---|---|---|---|
| | | | (EI+, | |
| Ex. No. | Structure | Compound Name | m/z, M⁺) | ¹H-NMR |
| Ex. 21 | 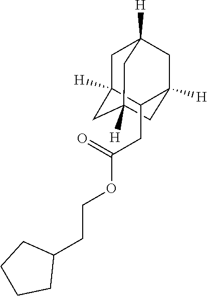 [SMILES: O=C(CC1[C@H]2C[C@@H]3C[C@@H](C[C@H]1C3)C2)OCCC1CCCC1] | 2-cyclopentylethyl 2- ((1R,3S,5r,7r)-adamantan- 2-yl)acetate | 290.22 | 1H NMR (300 MHz, DMSO-d6) δ 3.95-4.02 (m, J = 6.7 Hz, 2H), 2.43 (d, J = 7.7 Hz, 2H), 2.09-2.11 (m, J = 7.9 Hz, 1H), 1.89 − 1.40 (m, 23H), 1.14 − 1.02 (m, 2H). |
| Ex. 22 | 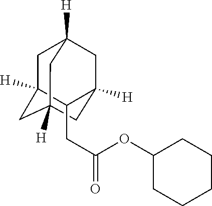 [SMILES: O=C(CC1[C@H]2C[C@@H]3C[C@@H](C[C@H]1C3)C2)OC1CCCCC1] | cyclohexyl 2- ((1R,3S,5r,7r)-adamantan- 2-yl)acetate | 276.21 | 1H NMR (300 MHz, DMSO-d6) δ 4.71 − 4.61 (m, 1H), 2.41 (d, J = 7.7 Hz, 2H), 2.19 − 2.06 (m, 1H), 1.86 − 1.61 (m, 16H), 1.49 (d, J = 12.6 Hz, 3H), 1.41 − 1.21 (m, 5H). |
| Ex. 23 | 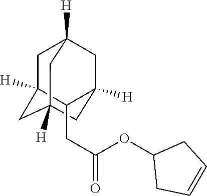 [SMILES: O=C(CC1[C@H]2C[C@@H]3C[C@@H](C[C@H]1C3)C2)OC1CC=CC1] | cyclopent-3-en-1-yl 2- ((1R,3S,5r,7r)-adamantan- 2-yl)acetate | 260.18 | 1H NMR (300 MHz, DMSO-d6) δ 5.72 (s, 2H), 5.33 − 5.20 (m, 1H), 2.76 − 2.62 (m, 2H), 2.40 (d, J = 7.7 Hz, 2H), 2.31 − 2.19 (m, 2H), 2.11 (d, J = 8.0 Hz, 1H), 1.86 − 1.67 (m, 9H), 1.64 (s, 2H), 1.48 (d, J = 12.3 Hz, 2H). |
| Ex. 24 | 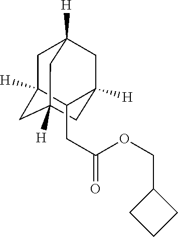 [SMILES: O=C(CC1[C@H]2C[C@@H]3C[C@@H](C[C@H]1C3)C2)OCC1CCC1] | cyclobutylmethyl 2- ((1R,3S,5r,7r)-adamantan- 2-yl)acetate | 262.19 | 1H NMR (300 MHz, DMSO-d6) δ 3.99 (d, J = 6.7 Hz, 2H), 2.62 − 2.52 (m, 1H), 2.44 (d, J = 7.7 Hz, 2H), 2.12 (t, J = 8.0 Hz, 1H), 2.04 − 1.62 (m, 18H), 1.49 (d, J = 12.5 Hz, 2H). |
| Ex. 25 | 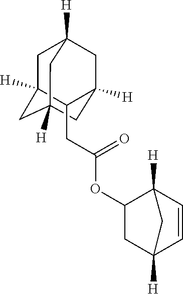 [SMILES: O=C(CC1[C@H]2C[C@@H]3C[C@@H](C[C@H]1C3)C2)OC1C[C@H]2C=C[C@@H]1C2] | (1S,4S)- bicyclo[2.2.1]hept-5-en-2- yl 2-((1R,3S,5S,7S)- adamantan-2-yl)acetate | 286.19 | 1H NMR (300 MHz, DMSO-d6) δ6.32- 6.34 (m, J = 5.8, 3.0 Hz, 1H), 5.90-5.92 (m, J = 5.8, 2.9 Hz, 1H), 5.12- 5.19 (m, J = 7.4, 3.3 Hz, 1H), 3.39 (d, J = 7.0 Hz, 1H), 3.06 (s, 1H), 2.81 (s, 1H), 2.39-2.41 (m, J = 8.3 Hz, 1H), 2.34 (d, J = 7.7 Hz, 2H), 2.00- 2.09 (m, J = 11.9, 8.1, 3.6 Hz, 2H), 1.83 (s, 3H), 1.81 − 1.66 (m, 9H), 1.62 (s, 2H), 1.48 (d, J = 12.7 Hz, 3H), 1.44 − 1.30 (m, 2H), 1.10 (t, J = 7.0 |
| | | | | Hz, 1H), 0.80 (d, J = |
| | | | | 12.4 Hz, 1H). |
| Ex. 26 | 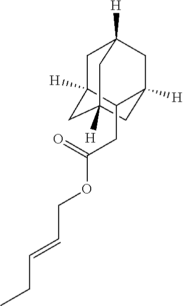 [SMILES: CC/C=C/COC(=O)CC1[C@H]2C[C@@H]3C[C@@H](C[C@H]1C3)C2] | (E)-pent-2-en-1-yl 2- ((1R,3S,5r,7r)-adamantan- 2-yl)acetate | 262.19 | 1H NMR (300 MHz, DMSO-d6) δ 5.87 − 5.72 (m, 1H), 5.61 − 5.44 (m, 1H), 4.52 − 4.43 (m, 2H), 2.45 (d, J = 7.7 Hz, 2H), 2.19 − 1.96 (m, 3H), 1.87 − 1.73 (m, 8H), 1.73 − 1.62 (m, 5H), 1.49 (d, J = 12.4 Hz, 2H), 0.95 (t, J = 7.4 Hz, 3H). |
| Ex. 27 | 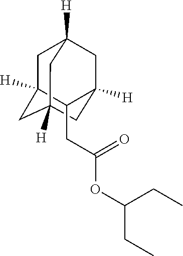 [SMILES: CCC(CC)OC(=O)CC1[C@H]2C[C@@H]3C[C@@H](C[C@H]1C3)C2] | pentan-3-yl 2- ((1R,3S,5r,7r)-adamantan- 2-yl)acetate | 264.21 | 1H NMR (300 MHz, DMSO-d6) δ 4.81 − 4.55 (m, 1H), 2.44 (d, J = 7.7 Hz, 2H), 2.13 (t, J = 7.9 Hz, 1H), 1.89 − 1.77 (m, 6H), 1.77 − 1.65 (m, 6H), 1.65 − 1.37 (m, 7H), 0.82 (t, J = 7.4 Hz, 6H). |
| Ex. 28 | 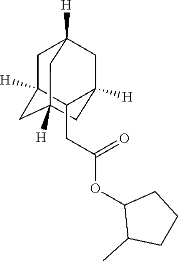 [SMILES: CC1CCCC1OC(=O)CC1[C@H]2C[C@@H]3C[C@@H](C[C@H]1C3)C2] | 2-methylcyclopentyl 2- ((1R,3S,5r,7r)-adamantan- 2-yl)acetate | 276.21 | 1H NMR (300 MHz, DMSO-d6) δ 4.99 (d, J = 4.7 Hz, 1H), 4.59- 4.60 (m, J = 6.6, 4.5 Hz, 1H), 2.40-2.42 (m, J = 7.8, 5.1 Hz, 4H), 2.12 (s, 2H), 1.84 (d, J = 6.4 Hz, 6H), 1.79 (s, 7H), 1.77 − 1.57 (m, 16H), 1.51 (t, J = 10.3 Hz, 6H), 1.35 − 1.11 (m, 2H),0.90-0.92 (m, J = 12.3, 6.8 Hz, 6H). |
| Ex. 29 | 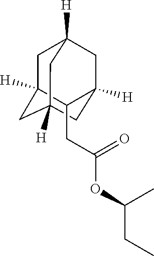 [SMILES: CC[C@H](C)OC(=O)CC1[C@H]2C[C@@H]3C[C@@H](C[C@H]1C3)C2] | (S)-sec-butyl 2- ((1R,3S,5S,7S)- adamantan-2-yl)acetate | 250.19 | 1H NMR (300 MHz, DMSO-d6) δ 4.79 − 4.65 (m, 1H), 2.46 − 2.37 (m, 2H), 2.12 (t, J = 7.9 Hz, 1H), 1.91 − 1.61 (m, 12H), 1.57 − 1.43 (m, 4H), 1.14 (d, J = 6.3 Hz, 3H), 0.83 (t, J = 7.4 Hz, 3H). |
| Ex. 30 | 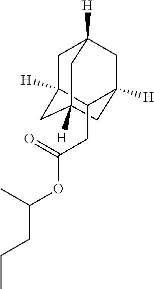 [SMILES: CCCC(C)OC(=O)CC1[C@H]2C[C@@H]3C[C@@H](C[C@H]1C3)C2] | pentan-2-yl 2- ((1R,3S,5r,7r)-adamantan- 2-yl)acetate | 264.21 | 1H NMR (300 MHz, DMSO-d6) δ 4.94 − 4.71 (m, 1H), 2.40 (d, J = 8.0 Hz, 2H), 2.11 (t, J = 8.0 Hz, 1H), 1.88 − 1.62 (m, 12H), 1.60 − 1.48 (m, 2H), 1.48 − 1.18 (m, 4H), 1.14 (d, J = 6.3 Hz, 3H), 0.86 (t, J = 7.2 Hz, 3H). |
| Ex. 31 | 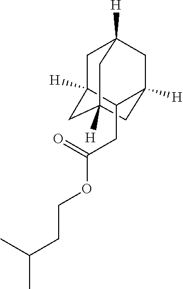 [SMILES: CC(C)CCOC(=O)CC1[C@H]2C[C@@H]3C[C@@H](C[C@H]1C3)C2] | isopentyl 2-((1R,3S,5r,7r)- adamantan-2-yl)acetate | 264.21 | 1H NMR (300 MHz, DMSO-d6) δ 4.04 (t, J = 6.7 Hz, 2H), 2.43 (d, J = 7.6 Hz, 2H), 2.11 (t, J = 8.0 Hz, 1H), 1.93 − 1.53 (m, 13H), 1.55 − 1.39 (m, 4H), 0.88 (d, J = 6.6 Hz, 6H). |
| Ex. 32 | 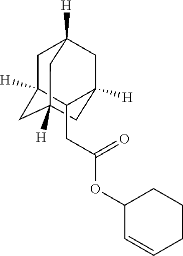 [SMILES: O=C(CC1[C@H]2C[C@@H]3C[C@@H](C[C@H]1C3)C2)OC1C=CCCC1] | cyclohex-2-en-1-yl 2- ((1R,3S,5r,7r)-adamantan- 2-yl)acetate | 274.19 | 1H NMR (300 MHz, DMSO-d6) δ 6.01-5.88 (m, 1H), 5.66-5.60 (m, 1H), 5.23 − 4.97 (m, 1H), 2.42 (d, J = 7.7 Hz, 2H), 2.21 − 1.92 (m, 3H), 1.89 − 1.55 (m, 20H), 1.56 − 1.43 (m, 4H), 1.40 (s, 7H), 1.33 − 1.18 (m, 5H), 1.00 − 0.91 (m, 2H), 0.90 − 0.78 (m, 7H). |
| Ex. 33 | 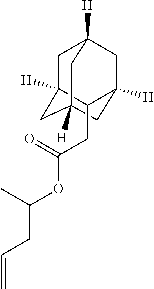 [SMILES: C=CCC(C)OC(=O)CC1[C@H]2C[C@@H]3C[C@@H](C[C@H]1C3)C2] | pent-4-en-2-yl 2- ((1R,3S,5r,7r)-adamantan- 2-yl)acetate | 262.19 | 1H NMR (300 MHz, DMSO-d6) δ 5.83 − 5.63 (m, 1H), 5.20 − 4.97 (m, 2H), 4.94 − 4.74 (m, 1H), 2.39 (d, J = 7.7 Hz, 2H), 2.31 − 2.22 (m, 2H), 2.10 (t, J = 7.9 Hz, 1H), 1.92 − 1.59 (m, 12H), 1.48 (d, J = 12.4 Hz, 2H), 1.15 (d, J = 6.3 Hz, 3H). |
| Ex. 34 | 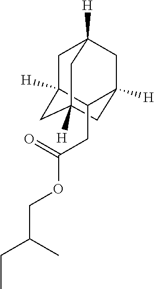 [SMILES: CCC(C)COC(=O)CC1[C@H]2C[C@@H]3C[C@@H](C[C@H]1C3)C2] | 2-methylbutyl 2- ((1R,3S,5r,7r)-adamantan- 2-yl)acetate | 264.21 | 1H NMR (300 MHz, DMSO-d6) δ 3.96 − 3.77 (m, 2H), 2.45 (d, J = 7.7 Hz, 2H), 2.12 (t, J = 8.0 Hz, 1H), 1.90 − 1.82 (m, 3H), 1.82 − 1.54 (m, 10H), 1.49 (d, J = 12.5 Hz, 2H), 1.45 − 1.28 (m, 1H), 1.25 − 1.07 (m, 1H), 0.92 − 0.81 (m, 6H). |
| Ex. 35 | 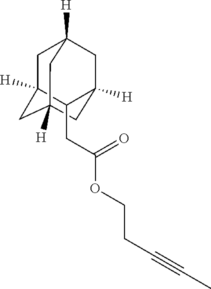 [SMILES: CC#CCCOC(=O)CC1[C@H]2C[C@@H]3C[C@@H](C[C@H]1C3)C2] | pent-3-yn-1-yl 2- ((1R,3S,5r,7r)-adamantan- 2-yl)acetate | 260.18 | 1H NMR (300 MHz, DMSO-d6) δ 4.04 (t, J = 6.6 Hz, 2H), 2.48 − 2.37 (m, 4H), 2.16 − 2.07 (m, 1H), 1.92 − 1.77 (m, 6H), 1.71 (m J = 13.3, 3.1 Hz, 9H), 1.49 (d, J = 12.5 Hz, 2H). |
| Ex. 36 | 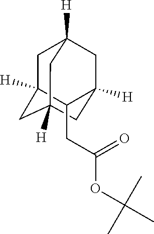 [SMILES: CC(C)(C)OC(=O)CC1[C@H]2C[C@@H]3C[C@@H](C[C@H]1C3)C2] | tert-butyl 2- ((1R,3S,5r,7r)-adamantan- 2-yl)acetate | 250.19 | 1H NMR (300 MHz, DMSO-d6) δ 2.32 (d, J = 7.7 Hz, 2H), 2.08 (t, J = 7.8 Hz, 1H), 1.87 − 1.57 (m, 13H), 1.49 (d, J = 12.3 Hz, 2H), 1.39 (s, 9H). |
| Ex. 37 | 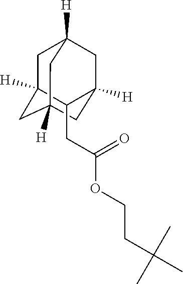 [SMILES: CC(C)(C)CCOC(=O)CC1[C@H]2C[C@@H]3C[C@@H](C[C@H]1C3)C2] | 3,3-dimethylbutyl 2- ((1R,3S,5r,7r)-adamantan- 2-yl)acetate | 278.22 | 1H NMR (300 MHz, DMSO-d6) δ 4.06 (t, J = 7.2 Hz, 2H), 2.42 (d, J = 7.7 Hz, 2H), 2.19 − 2.05 (m, 1H), 1.90 − 1.59 (m, 12H), 1.55 − 1.42 (m, 4H), 0.91 (s, 9H). |
| Ex. 38 | 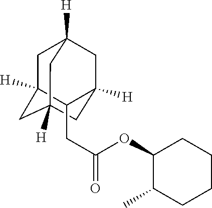 [SMILES: C[C@H]1CCCC[C@@H]1OC(=O)CC1[C@H]2C[C@@H]3C[C@@H](C[C@H]1C3)C2] | (1S,2S)-2- methylcyclohexyl 2- ((1R,3S,5S,7S)- adamantan-2-yl)acetate | 290.22 | 1H NMR (300 MHz, DMSO-d6) δ 4.36 − 4.24 (m, 1H), 2.43 (d, J = 7.8 Hz, 2H), 2.19 − 2.07 (m, 1H), 1.91 − 1.62 (m, 13H), 1.62 − 1.43 (m, 4H), 1.35 − 0.98 (m, 4H), 0.83 (d, J = 6.5 Hz, 3H). |
| Ex. 39 | 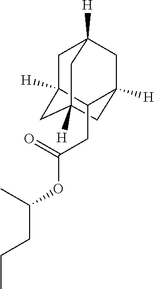 [SMILES: CCC[C@H](C)OC(=O)CC1[C@H]2C[C@@H]3C[C@@H](C[C@H]1C3)C2] | (S)-pentan-2-yl 2- ((1R,3S,5S,7S)- adamantan-2-yl)acetate | 264.21 | 1H NMR (300 MHz, DMSO-d6) δ 4.86 − 4.75 (m, 1H), 2.40 (d, J = 7.8 Hz, 2H), 2.17 − 2.05 (m, 1H), 1.90 − 1.59 (m, 12H), 1.56 − 1.38 (m, 4H), 1.35 − 1.21 (m, 2H), 1.14 (d, J = 6.2 Hz, 3H), 0.91 − 0.80 (m, 3H). |
| Ex. 40 | 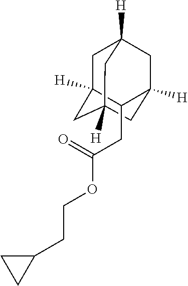 [SMILES: O=C(CC1[C@H]2C[C@@H]3C[C@@H](C[C@H]1C3)C2)OCCC1CC1] | 2-cyclopropylethyl 2- ((1R,3S,5r,7r)-adamantan- 2-yl)acetate | 262.19 | 1H NMR (300 MHz, DMSO-d6) δ 4.12 − 3.99 (m, 2H), 2.44 (d, J = 7.7 Hz, 2H), 2.13 (t, J = 8.0 Hz, 1H), 1.89 − 1.72 (m, 8H), 1.71 − 1.61 (m, 5H), 1.56 − 1.40 (m, 4H), 0.78 − 0.62 (m, 1H), 0.45 − 0.35 (m, 2H), 0.11 − 0.02 (m, 2H). |
| Ex. 41 | 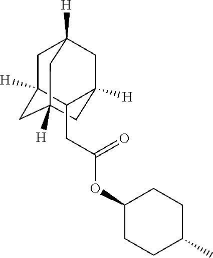 [SMILES: C[C@H]1CC[C@H](OC(=O)CC2[C@H]3C[C@@H]4C[C@@H](C[C@H]2C4)C3)CC1] | (1r,4R)-4- methylcyclohexyl 2- ((1R,3S,5R,7R)- adamantan-2-yl)acetate | 290.22 | 1H NMR (300 MHz, DMSO-d6) δ 4.63 − 4.48 (m, 1H), 2.39 (d, J = 7.7 Hz, 2H), 2.16 − 2.04 (m, 1H), 1.91 − 1.59 (m, 16H), 1.54 − 1.43 (m, 2H), 1.41 − 1.21 (m, 3H), 1.08 − 0.92 (m, 2H), 0.86 (d, J = 6.5 Hz, 3H). |
| Ex. 42 | 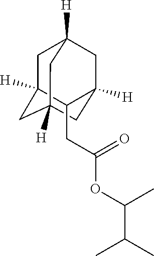 [SMILES: CC(C)C(C)OC(=O)CC1[C@H]2C[C@@H]3C[C@@H](C[C@H]1C3)C2] | 3-methylbutan-2-yl 2- ((1R,3S,5r,7r)-adamantan- 2-yl)acetate | 264.21 | 1H NMR (300 MHz, DMSO-d6) δ 4.67 − 4.55 (m, 1H), 2.54 − 2.34 (m, 3H), 1.90 − 1.55 (m, 11H), 1.53 − 1.44 (m, 2H), 1.13 − 1.03 (m, 3H), 0.90 − 0.78 (m, 6H). |
| Ex. 43 | 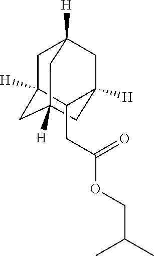 [SMILES: CC(C)COC(=O)CC1[C@H]2C[C@@H]3C[C@@H](C[C@H]1C3)C2] | isobutyl 2-((1R,3S,5r,7r)- adamantan-2-yl)acetate | 250.19 | 1H NMR (300 MHz, DMSO-d6) δ 3.80 (d, J = 6.6 Hz, 2H), 2.45 (d, J = 7.7 Hz, 2H), 2.18 − 2.07 (m, 1H), 1.90 − 1.61 (m, 13H), 1.54 − 1.44 (m, 2H), 0.88 (d, J = 6.7 Hz, 6H). |
| Ex. 44 | 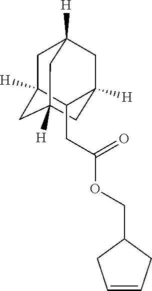 [SMILES: O=C(CC1[C@H]2C[C@@H]3C[C@@H](C[C@H]1C3)C2)OCC1CC=CC1] | cyclopent-3-en-1- ylmethyl 2-((1R,3S,5r,7r)- adamantan-2-yl)acetate | 274.19 | 1H NMR (300 MHz, DMSO-d6) δ 5.67 (s, 2H), 3.92 (d, J = 7.0 Hz, 2H), 2.47 − 2.33 (m, 4H), 2.17 − 1.96 (m, 3H), 1.90 − 1.61 (m, 12H), 1.54 − 1.43 (m, 2H). |
| Ex. 45 | 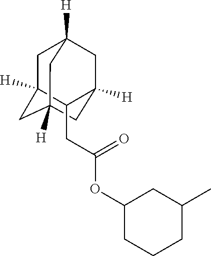 [SMILES: CC1CCCC(OC(=O)CC2[C@H]3C[C@@H]4C[C@@H](C[C@H]2C4)C3)C1] | 2-methylcyclohexyl 2- ((1R,3S,5r,7r)-adamantan- 2-yl)acetate | 290.22 | 1H NMR (300 MHz, DMSO-d6) δ 5.04 − 4.46 (m, 1H), 2.47 − 2.35 (m, 2H), 2.20-2.02 (m, 1H), 1.90 − 1.57 (m, 15H), 1.57 − 1.36 (m, 5H), 1.34 − 1.05 (m, 2H), 1.03 − 0.71 (m, 5H). |
| Ex. 46 | 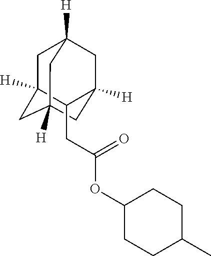 [SMILES: CC1CCC(OC(=O)CC2[C@H]3C[C@@H]4C[C@@H](C[C@H]2C4)C3)CC1] | 4-methylcyclohexyl 2- ((1R,3S,5r,7r)-adamantan- 2-yl)acetate | 290.22 | 1H NMR (300 MHz, DMSO-d6) δ 4.92 − 4.48 (m, 1H), 2.46 − 2.35 (m, 2H), 2.19 − 2.04 (m, 1H), 1.92 − 1.59 (m, 15H), 1.57 − 0.94 (m, 9H), 0.92 − 0.82 (m, 3H). |
| Ex. 47 | 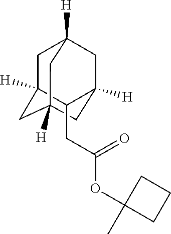 [SMILES: CC1(OC(=O)CC2[C@H]3C[C@@H]4C[C@@H](C[C@H]2C4)C3)CCC1] | 1-methylcyclobutyl 2- ((1R,3S,5r,7r)-adamantan- 2-yl)acetate | 262.19 | 1H NMR (300 MHz, DMSO-d6) δ 2.35 (d, J = 7.7 Hz, 2H), 2.27 − 1.98 (m, 5H), 1.91 − 1.60 (m, 13H), 1.54 − 1.39 (m, 5H). |
| Ex. 48 | 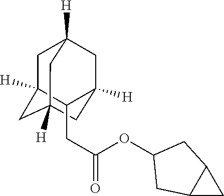 [SMILES: O=C(CC1[C@H]2C[C@@H]3C[C@@H](C[C@H]1C3)C2)OC1CC2CC2C1] | bicyclo[3.1.0]hexan-3-yl 2-((1R,3S,5r,7r)- adamantan-2-yl)acetate | 274.19 | 1H NMR (300 MHz, DMSO-d6) δ 5.18 − 5.06 (m, 1H), 2.44 − 2.32 (m, 2H), 2.24 − 2.03 (m, 4H), 1.90 − 1.57 (m, 14H), 1.54 − 1.42 (m, 2H), 1.37 − 1.23 (m, 2H), 0.53 − 0.41 (m, 1H), 0.35 − 0.26 (m, 1H). |
| Ex. 49 | 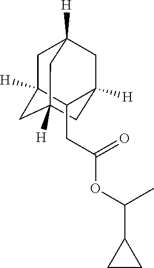 [SMILES: CC(OC(=O)CC1[C@H]2C[C@@H]3C[C@@H](C[C@H]1C3)C2)C1CC1] | 1-cyclopropylethyl 2- ((1R,3S,5r,7r)-adamantan- 2-yl)acetate | 262.19 | 1H NMR (300 MHz, DMSO-d6) δ 4.33 − 4.17 (m, 1H), 2.41 (d, J = 7.7 Hz, 2H), 2.17 − 2.06 (m, 1H), 1.91 − 1.62 (m, 12H), 1.49 (d, J = 12.5 Hz, 2H), 1.22 (d, J = 6.3 Hz, 3H), 1.06 − 0.90 (m, 1H), 0.56 − 0.36 (m, 2H), 0.33 − 0.17 (m, 2H). |
| Ex. 50 | 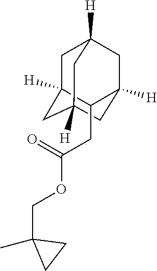 [SMILES: CC1(COC(=O)CC2[C@H]3C[C@@H]4C[C@@H](C[C@H]2C4)C3)CC1] | (1- methylcyclopropyl)methyl 2-((1R,3S,5r,7r)- adamantan-2-yl)acetate | 262.19 | 1H NMR (300 MHz, DMSO-d6) δ 3.82 (s, 2H), 2.46 (d, J = 7.7 Hz, 2H), 2.18 − 2.07 (m, 1H), 1.92 − 1.61 (m, 7H), 1.49 (d, J = 12.5 Hz, 2H), 1.06 (s, 3H), 0.48 − 0.41 (m, 2H), 0.36 − 0.29 (m, 2H). |
| Ex. 51 | 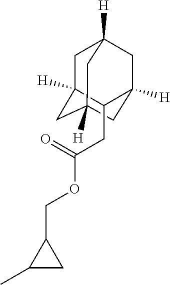 [SMILES: CC1CC1COC(=O)CC1[C@H]2C[C@@H]3C[C@@H](C[C@H]1C3)C2] | (1- methylcyclopropyl)methyl 2-((1R,3S,5r,7r)- adamantan-2-yl)acetate | 262.19 | 1H NMR (300 MHz, DMSO-d6) δ 4.26 − 3.73 (m, 2H), 2.44 (dd, J = 7.7, 2.8 Hz, 2H), 2.14 (d, J = 8.0 Hz, 1H), 1.93 − 1.58 (m, 12H), 1.58 − 1.44 (m, 2H), 1.00 (dd, J = 9.3, 6.0 Hz, 3H), 0.93 − 0.60 (m, 2H), 0.45 − 0.16 (m, 1H). |
| Ex. 52 | 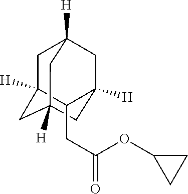 [SMILES: O=C(CC1[C@H]2C[C@@H]3C[C@@H](C[C@H]1C3)C2)OC1CC1] | cyclopropyl 2- ((1R,3S,5r,7r)-adamantan- 2-yl)acetate | 234.16 | 1H NMR (300 MHz, DMSO-d6) δ 4.16 − 3.91 (m, 1H), 2.42 − 2.28 (m, 2H), 2.14 − 1.96 (m, 1H), 1.86 − 1.71 (m, 8H), 1.68 − 1.36 (m, 7H), 0.74 − 0.63 (m, 2H), 0.63 − 0.49 (m, 2H). |
| Ex. 53 | 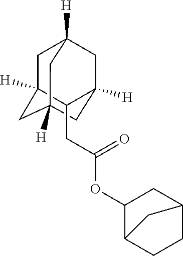 [SMILES: O=C(CC1[C@H]2C[C@@H]3C[C@@H](C[C@H]1C3)C2)OC1CC2CCC1C2] | bicyclo[2.2.1]heptan-2-yl 2-((1R,3S,5r,7r)- adamantan-2-yl)acetate | 288.21 | 1H NMR (300 MHz, DMSO-d6) δ 4.90 − 4.45 (m, 1H), 2.47 − 2.31 (m, 3H), 2.29 − 2.04 (m, 2H), 2.02 − 1.61 (m, 12H), 1.60 − 1.01 (m, 8H), 0.97 − 0.81 (m, 1H). |
| Ex. 54 | 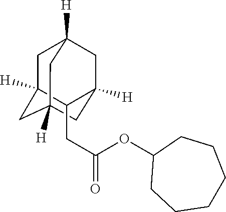 [SMILES: O=C(CC1[C@H]2C[C@@H]3C[C@@H](C[C@H]1C3)C2)OC1CCCCCC1] | cycloheptyl 2- ((1R,3S,5r,7r)-adamantan- 2-yl)acetate | 290.22 | 1H NMR (300 MHz, DMSO-d6) δ 4.90 − 4.76 (m, 1H), 2.44 − 2.35 (m, 2H), 2.17 − 2.06 (m, 1H), 1.88 − 1.85 (m, 1H), 1.85 − 1.61 (m, 14H), 1.61 − 1.44 (m, 9H), 1.43 − 1.37 (m, 2H). |
| Ex. 55 | 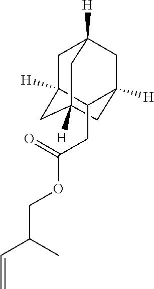 [SMILES: C=CC(C)COC(=O)CC1[C@H]2C[C@@H]3C[C@@H](C[C@H]1C3)C2] | 2-methylbut-3-en-1-yl 2- ((1R,3S,5r,7r)-adamantan- 2-yl)acetate | 262.19 | 1H NMR (300 MHz, DMSO-d6) δ 5.83 − 5.66 (m, 1H), 5.13 − 4.97 (m, 2H), 4.01 − 3.83 (m, 2H), 2.52 − 2.40 (m, 3H), 2.17 − 2.09 (m, 1H), 1.88 − 1.82 (m, 2H), 1.86 − 1.62 (m, 11H), 1.54 − 1.44 (m, 2H), 1.02 − 0.94 (m, 2H). |
| Ex. 56 | 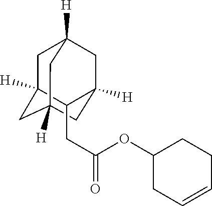 [SMILES: O=C(CC1[C@H]2C[C@@H]3C[C@@H](C[C@H]1C3)C2)OC1CC=CCC1] | cyclohex-3-en-1-yl 2- ((1R,3S,5r,7r)-adamantan- 2-yl)acetate | 274.19 | 1H NMR (300 MHz, DMSO-d6) δ 5.73 − 5.61 (m, 1H), 5.61 − 5.50 (m, 1H), 4.97 − 4.83 (m, 1H), 2.46 − 2.38 (m, 2H), 2.38 − 2.26 (m, 1H), 2.17 − 2.02 (m, 3H), 2.05 − 1.99 (m, 1H), 1.88 − 1.55 (m, 14H), 1.55 − 1.43 (m, 2H). |
| Ex. 57 | 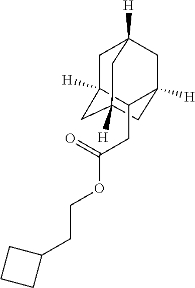 [SMILES: O=C(CC1[C@H]2C[C@@H]3C[C@@H](C[C@H]1C3)C2)OCCC1CCC1] | 2-cyclobutylethyl 2- ((1R,3S,5r,7r)-adamantan- 2-yl)acetate | 276.21 | 1H NMR (300 MHz, DMSO-d6) δ 3.98 − 3.88 (m, 2H), 2.46 − 2.38 (m, 2H), 2.38 − 2.22 (m, 1H), 2.16 − 2.06 (m, 1H), 2.06 − 1.91 (m, 2H), 1.87 − 1.73 (m, 8H), 1.78 − 1.64 (m, 5H), 1.68 − 1.61 (m, 4H), 1.64 − 1.55 (m, 1H), 1.54 − 1.44 (m, 2H). |
| Ex. 58 | 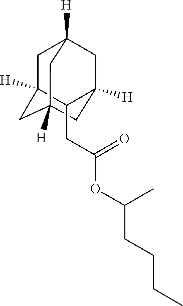 [SMILES: CCCCC(C)OC(=O)CC1[C@H]2C[C@@H]3C[C@@H](C[C@H]1C3)C2] | hexan-2-yl 2- ((1R,3S,5r,7r)-adamantan- 2-yl)acetate | 278.22 | 1H NMR (300 MHz, DMSO-d6) δ 4.87 − 4.71 (m, 1H), 2.45 − 2.36 (m, 2H), 2.17 − 2.05 (m, 1H), 1.88 − 1.61 (m, 13H), 1.54 − 1.42 (m, 3H), 1.29 − 1.23 (m, 2H), 1.32 − 1.19 (m, 2H), 1.23 − 1.10 (m, 3H), 0.91 − 0.79 (m, 3H). |
| Ex. 59 | 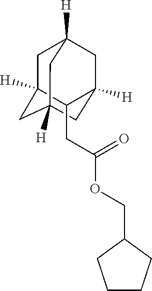 [SMILES: O=C(CC1[C@H]2C[C@@H]3C[C@@H](C[C@H]1C3)C2)OCC1CCCC1] | cyclobutylmethyl 2- ((1R,3S,5r,7r)-adamantan- 2-yl)acetate | 276.21 | 1H NMR (300 MHz, DMSO-d6) δ 3.94 − 3.86 (m, 2H), 2.48 − 2.37 (m, 2H), 2.22 − 2.03 (m, 2H), 1.89 − 1.76 (m, 6H), 1.76 − 1.62 (m, 8H), 1.62 − 1.44 (m, 6H), 1.29 − 1.12 (m, 2H). |
| Ex. 60 | 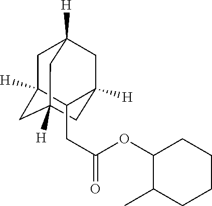 [SMILES: CC1CCCCC1OC(=O)CC1[C@H]2C[C@@H]3C[C@@H](C[C@H]1C3)C2] | 2-methylcyclohexyl 2- ((1R,3S,5r,7r)-adamantan- 2-yl)acetate | 290.22 | 1H NMR (300 MHz, DMSO-d6) δ 2.50 − 2.39 (m, 2H), 2.18 − 2.10 (m, 2H), 1.89 − 1.57 (m, 15H), 1.57 − 1.47 (m, 2H), 1.51 − 1.35 (m, 3H), 1.39 − 1.13 (m, 2H), 1.28 − 1.22 (m, 2H), 0.87 − 0.77 (m, 2H). |
| Ex. 61 | 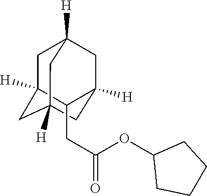 [SMILES: O=C(CC1[C@H]2C[C@@H]3C[C@@H](C[C@H]1C3)C2)OC1CCCC1] | cyclopentyl 2- ((1R,3S,5r,7r)-adamantan- 2-yl)acetate | 262.19 | 1H NMR (300 MHz, DMSO-d6) δ 4.05 − 3.95 (m, 2H), 2.50 − 2.29 (m, 2H), 2.17 − 2.06 (m, 1H), 1.88 − 1.61 (m, 10H), 1.60 − 1.43 (m, 4H), 1.30 − 1.23 (m, 5H), 0.91 − 0.81 (m, 2H). |
| Ex. 62 | 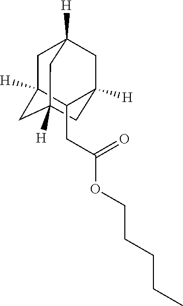 [SMILES: CCCCCOC(=O)CC1[C@H]2C[C@@H]3C[C@@H](C[C@H]1C3)C2] | pentyl 2-((1R,3S,5r,7r)- adamantan-2-yl)acctatc | 264.21 | 1H NMR (300 MHz, DMSO-d6) δ 4.05 − 3.95 (m, 2H), 2.48 − 2.39 (m, 2H), 2.17 − 2.06 (m, 1H), 1.81 − 1.76 (m, 3H), 1.76 − 1.62 (m, 5H), 1.61 − 1.44 (m, 5H), 1.32 − 1.26 (m, 5H), 1.33 − 1.22 (m, 2H), 0.92 − 0.81 (m, 3H). |
| Ex. 63 | 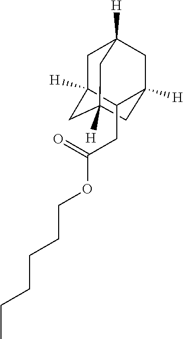 [SMILES: CCCCCCOC(=O)CC1[C@H]2C[C@@H]3C[C@@H](C[C@H]1C3)C2] | hexyl 2-((1R,3S,5r,7r)- adamantan-2-yl)acetate | 278.22 | 1H NMR (300 MHz, DMSO-d6) δ 4.05 − 3.95 (m, 2H), 2.47 − 2.39 (m, 2H), 2.17 − 2.06 (m, 1H), 1.89 − 1.75 (m, 5H), 1.80 − 1.67 (m, 5H), 1.67 − 1.62 (m, 2H), 1.62 − 1.43 (m, 4H), 1.37 − 1.20 (m, 6H), 0.94 − 0.79 (m, 3H). |
| Ex. 64 | 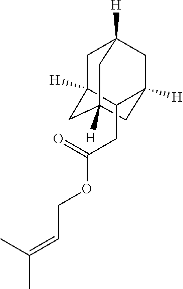 [SMILES: CC(C)=CCOC(=O)CC1[C@H]2C[C@@H]3C[C@@H](C[C@H]1C3)C2] | 3-methylbut-2-en-1-yl 2- ((1R,3S,5r,7r)-adamantan- 2-yl)acctatc | 262.19 | 1H NMR (300 MHz, DMSO-d6) δ 5.35 − 5.23 (m, 1H), 4.55 − 4.46 (m, 2H), 2.47 − 2.38 (m, 2H), 2.17 − 2.05 (m, 1H), 1.89 − 1.58 (m, 18H), 1.54 − 1.43 (m, 2H). |
| Ex. 65 | 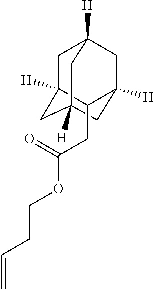 [SMILES: C=CCCOC(=O)CC1[C@H]2C[C@@H]3C[C@@H](C[C@H]1C3)C2] | but-3-en-1-yl 2- ((1R,3S,5r,7r)-adamantan- 2-yl)acetate | 248.18 | 1H NMR (300 MHz, DMSO-d6) δ 5.87 − 5.67 (m, 1H), 5.16 − 5.00 (m, 2H), 4.12 − 4.02 (m, 2H), 2.50 − 2.38 (m, 2H), 2.38 − 2.26 (m, 2H), 2.17 − 2.05 (m, 1H), 1.89 − 1.61 (m, 12H), 1.54 − 1.43 (m, 2H). |
| Ex. 66 | 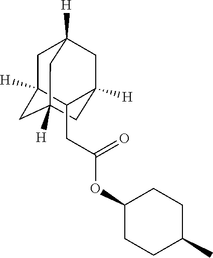 [SMILES: C[C@H]1CC[C@@H](OC(=O)CC2[C@H]3C[C@@H]4C[C@@H](C[C@H]2C4)C3)CC1] | (1s,4S)-4- methylcyclohexyl 2- ((1R,3S,5R,7R)- adamantan-2-yl)acctatc | 290.22 | 1H NMR (300 MHz, DMSO-d6) δ 2.43 (d, J = 7.8 Hz, 2H), 2.14 (t, J = 8.0 Hz, 1H), 1.90 − 1.77 (m, 6H), 1.77 − 1.63 (m, 8H), 1.58 − 1.43 (m, 6H), 1.16 (q, J = 13.0, 11.8 Hz, 2H), 0.88 (d, J = 6.1 Hz, 3H) |
| Ex. 67 | 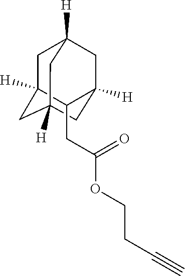 [SMILES: C#CCCOC(=O)CC1[C@H]2C[C@@H]3C[C@@H](C[C@H]1C3)C2] | but-3-yn-1-yl 2- ((1R,3S,5r,7r)-adamantan- 2-yl)acetate | 246.16 | 1H NMR (300 MHz, DMSO-d6) δ 4.08 (t, J = 6.4 Hz, 3H), 2.85 (t, J = 2.7 Hz, 1H), 2.51 (d, J = 2.9 Hz, 3H), 2.45 (d, J = 7.5 Hz, 5H), 2.13 (t, J = 7.8 Hz, 1H), 1.89 − 1.65 (m, 19H), 1.57 (s, 1H), 1.54 − 1.44 (m, 5H). |
| Ex. 68 | 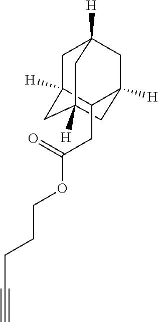 [SMILES: C#CCCCOC(=O)CC1[C@H]2C[C@@H]3C[C@@H](C[C@H]1C3)C2] | pent-4-yn-1-yl 2- ((1R,3S,5r,7r)-adamantan- 2-yl)acetate | 260.18 | 1H NMR (300 MHz, DMSO-d6) δ 4.07 (t, J = 6.4 Hz, 2H), 2.81 (t, J = 2.7 Hz, 1H), 2.45 (d, J = 7.7 Hz, 2H), 2.28 − 2.06 (m, 3H), 1.88 − 1.82 (m, 2H), 1.82 − 1.62 (m, 11H), 1.55 − 1.44 (m, 2H). |
| Ex. 69 | 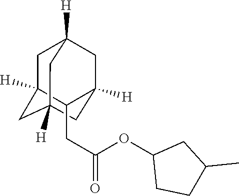 [SMILES: CC1CCC(OC(=O)CC2[C@H]3C[C@@H]4C[C@@H](C[C@H]2C4)C3)C1] | 3-methylcyclopentyl 2- ((1R,3S,5r,7r)-adamantan- 2-yl)acetate | 276.21 | 1H NMR (300 MHz, DMSO-d6) δ 5.14 − 4.92 (m, 1H), 2.39 (dd, J = 7.7, 3.1 Hz, 2H), 2.23 − 1.76 (m, 11H), 1.72 (d, J = 14.5 Hz, 5H), 1.67 − 1.61 (m, 2H), 1.60 − 1.44 (m, 2H), 1.41 − 0.93 (m, 5H). |
| Ex. 70 | 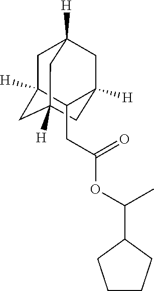 [SMILES: CC(OC(=O)CC1[C@H]2C[C@@H]3C[C@@H](C[C@H]1C3)C2)C1CCCC1] | 1-cyclopentylethyl 2- ((1R,3S,5r,7r)-adamantan- 2-yl)acetate | 290.22 | 1H NMR (300 MHz, DMSO-d6) δ 4.67 (dq, J = 7.7, 6.2 Hz, 1H), 2.48 − 2.34 (m, 2H), 2.12 (t, J = 8.0 Hz, 1H), 1.97 (h, J = 8.1 Hz, 1H), 1.87 − 1.76 (m, 6H), 1.69 (t, J = 12.6 Hz, 8H), 1.61 − 1.43 (m, 6H), 1.30 − 1.10 (m, 5H). |
| Ex. 71 | 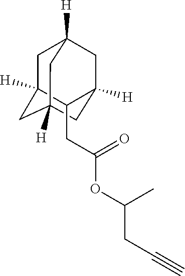 [SMILES: C#CCC(C)OC(=O)CC1[C@H]2C[C@@H]3C[C@@H](C[C@H]1C3)C2] | pent-4-yn-2-yl 2- ((1R,3S,5r,7r)-adamantan- 2-yl)acetate | 260.18 | 1H NMR (300 MHz, DMSO-d6) δ 4.86 (p, J = 6.1 Hz, 1H), 2.87 (t, J = 2.7 Hz, 1H), 2.49 − 2.38 (m, 4H), 2.13 (t, J = 7.9 Hz, 1H), 1.76 (d, J = 11.8 Hz, 4H), 1.69 (s, 4H), 1.49 (d, J = 12.4 Hz, 2H), 1.22 (d, J = 6.3 Hz, 3H). |
| Ex. 72 | 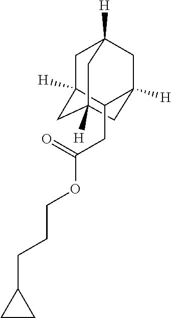 [SMILES: O=C(CC1[C@H]2C[C@@H]3C[C@@H](C[C@H]1C3)C2)OCCCC1CC1] | 3-cyclopropylpropyl 2- ((1R,3S,5r,7r)-adamantan- 2-yl)acetate | 276.21 | 1H NMR (300 MHz, DMSO-d6) δ 4.03 (t, J = 6.6 Hz, 2H), 2.43 (d, J = 7.7 Hz, 2H), 2.11 (t, J = 8.0 Hz, 1H), 1.84 (d, J = 5.7 Hz, 3H), 1.79 (d, J = 3.5 Hz, 3H), 1.76 − 1.67 (m, 5H), 1.67 − 1.57 (m, 4H), 1.48 (d, J = 12.3 Hz, 2H), 1.21 (q, J = 7.1 Hz, 2H), 0.75 − 0.57 (m, 1H), 0.45 − 0.31 (m, 2H). |
| Ex. 73 | 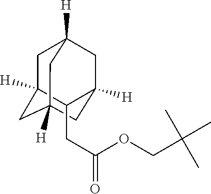 [SMILES: CC(C)(C)COC(=O)CC1[C@H]2C[C@@H]3C[C@@H](C[C@H]1C3)C2] | neopentyl 2- ((1R,3S,5r,7r)-adamantan- 2-yl)acetate | 264.21 | 1H NMR (300 MHz, DMSO-d6) δ 3.71 (s, 2H), 2.47 (d, J = 7.8 Hz, 4H), 2.13 (t, J = 8.0 Hz, 1H), 1.90 − 1.66 (m, 11H), 1.49 (d, J = 12.5 Hz, 2H), 0.90 (s, 8H). |
| Ex. 74 | 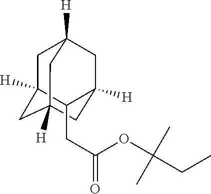 [SMILES: CCC(C)(C)OC(=O)CC1[C@H]2C[C@@H]3C[C@@H](C[C@H]1C3)C2] | tert-pentyl 2- ((1R,3S,5r,7r)-adamantan- 2-yl)acetate | 264.21 | 1H NMR (300 MHz, DMSO-d6) δ 2.34 (d, J = 7.7 Hz, 2H), 2.09 (t, J = 8.0 Hz, 1H), 1.87 − 1.76 (m, 6H), 1.70 (td, J = 10.4, 9.1, 4.6 Hz, 8H), 1.49 (d, J = 12.3 Hz, 2H), 1.36 (s, 6H), 0.83 (t, J = 7.5 Hz, 3H). |
| Ex. 75 | 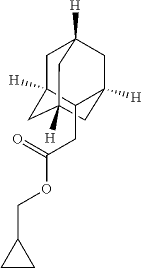 [SMILES: O=C(CC1[C@H]2C[C@@H]3C[C@@H](C[C@H]1C3)C2)OCC1CC1] | cyclopropylmethyl 2- ((1R,3S,5r,7r)-adamantan- 2-yl)acetate | 248.18 | 1H NMR (300 MHz, DMSO-d6) δ 3.85 (d, J = 7.2 Hz, 2H), 2.45 (d, J = 7.6 Hz, 2H), 2.13 (t, J = 7.8 Hz, 1H), 1.89 − 1.71 (m, 8H), 1.68 (dt, J = 8.1, 3.1 Hz, 4H), 1.49 (d, J = 12.5 Hz, 2H), 1.17 − 0.97 (m, 1H), 0.56 − 0.42 (m, 2H), 0.30 − 0.19 (m, 2H). |
| Ex. 76 | 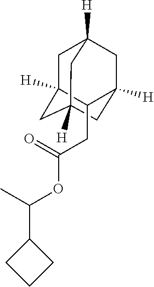 [SMILES: CC(OC(=O)CC1[C@H]2C[C@@H]3C[C@@H](C[C@H]1C3)C2)C1CCC1] | 2-cyclobutylethyl 2- ((1R,3S,5r,7r)-adamantan- 2-yl)acetate | 276.21 | 1H NMR (300 MHz, DMSO-d6) δ 4.77 (p, J = 6.4 Hz, 1H), 2.49 − 2.33 (m, 3H), 1.98 − 1.63 (m, 18H), 1.49 (d, J = 12.5 Hz, 2H), 1.04 (d, J = 6.3 Hz, 3H). |
| Ex. 77 | 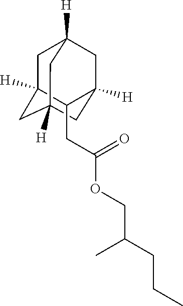 [SMILES: CCCC(C)COC(=O)CC1[C@H]2C[C@@H]3C[C@@H](C[C@H]1C3)C2] | 2-methylpentyl 2- ((1R,3S,5r,7r)-adamantan- 2-yl)acetate | 278.22 | 1H NMR (300 MHz, DMSO-d6) δ 3.96-3.73 (m, 2H), 2.54 − 2.41 (m, 3H), 2.13 (t, J = 7.9 Hz, 1H), 1.90-1.63 (m, 10H), 1.50 (d, J = 12.6 Hz, 2H), 1.36 − 1.18 (m, 2H), 1.18-1.04 (m, 1H), 0.87 (dd, J = 6.9, 2.7 Hz, 4H). |
| Ex. 78 | 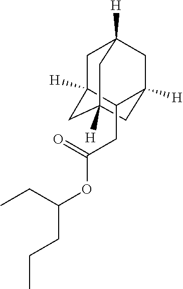 [SMILES: CCCC(CC)OC(=O)CC1[C@H]2C[C@@H]3C[C@@H](C[C@H]1C3)C2] | Hexan-3-yl 2- ((1R,3S,5r,7r)-adamantan- 2-yl)acetate | 278.22 | 1H NMR (300 MHz, DMSO-d6) δ 4.73 (ddd, J = 12.6, 6.8, 5.3 Hz, 1H), 2.43 (d, J = 7.8 Hz, 2H), 2.12 (t, J = 8.0 Hz, 1H), 1.89 − 1.71 (m, 7H), 1.71 − 1.62 (m, 4H), 1.62 − 1.36 (m, 6H), 1.36 − 1.12 (m, 2H), 0.83 (dt, J = 14.6, 7.4 Hz, 6H). |
| Ex. 79 | 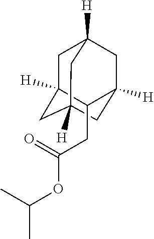 [SMILES: CC(C)OC(=O)CC1[C@H]2C[C@@H]3C[C@@H](C[C@H]1C3)C2] | isopropyl 2- ((1R,3S,5r,7r)-adamantan- 2-yl)acetate | 236.18 | 1H NMR (300 MHz, DMSO-d6) δ 4.94 − 4.81 (m, 1H), 2.41 − 2.25 (m, 2H), 2.11 (s, 1H), 1.87 − 1.61 (m, 13H), 1.55 − 1.44 (m, 2H), 1.18 − 1.15 (m, 5H). |
| Ex. 80 | 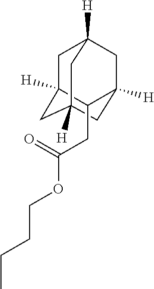 [SMILES: CCCCOC(=O)CC1[C@H]2C[C@@H]3C[C@@H](C[C@H]1C3)C2] | butyl 2-((1R,3S,5r,7r)- adamantan-2-yl)acetate | 250.19 | 1H NMR (300 MHz, DMSO-d6) δ 4.06 − 3.96 (m, 2H), 2.48 − 2.39 (m, 2H), 2.17 − 2.06 (m, 1H), 1.90 − 1.71 (m, 3H), 1.80 (s, 4H), 1.77 − 1.62 (m, 5H), 1.61 − 1.48 (m, 3H), 1.52 − 1.43 (m, 1H), 1.41 − 1.23 (m, 2H), 0.94 − 0.83 (m, 3H). |
| Ex. 81 | 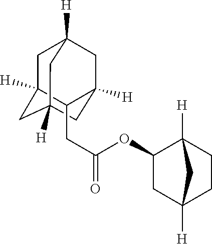 [SMILES: O=C(CC1[C@H]2C[C@@H]3C[C@@H](C[C@H]1C3)C2)O[C@@H]1C[C@H]2CC[C@@H]1C2] | (1R,2R,4S)- bicyclo[2.2.1]heptan-2-yl 2-((1R,3S,5R,7R)- adamantan-2-yl)acetate | 288.21 | 1H NMR (300 MHz, DMSO-d6) δ 4.54 − 4.46 (m, 1H), 2.43 − 2.34 (m, 2H), 2.28 − 2.16 (m, 2H), 2.16 − 2.07 (m, 1H), 1.84 (s, 2H), 1.82 − 1.61 (m, 11H), 1.54 − 1.45 (m, 3H), 1.45 − 1.37 (m, 2H), 1.37 − 1.26 (m, 1H), 1.18 − 1.04 (m, 3H). |
| Ex. 82 | 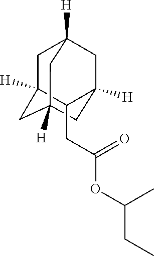 [SMILES: CCC(C)OC(=O)CC1[C@H]2C[C@@H]3C[C@@H](C[C@H]1C3)C2] | butan-2-yl 2- ((1R,3S,5r,7r)-adamantan- 2-yl)acetate | 250.19 | 1H NMR (300 MHz, DMSO-d6) δ 4.81 − 4.65 (m, 1H), 2.48 − 2.37 (m, 2H), 2.18 − 2.06 (m, 1H), 1.92 − 1.72 (m, 7H), 1.72 − 1.60 (m, 5H), 1.59 − 1.43 (m, 4H), 1.18 − 1.10 (m, 3H), 0.89 − 0.78 (m, 3H). |
| Ex. 83 | 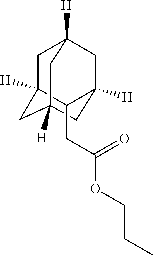 [SMILES: CCCOC(=O)CC1[C@H]2C[C@@H]3C[C@@H](C[C@H]1C3)C2] | propyl 2-((1R,3S,5r,7r)- adamantan-2-yl)acetate | 236.18 | 1H NMR (300 MHz, DMSO-d6) δ 4.02 − 3.91 (m, 2H), 2.49 − 2.40 (m, 2H), 2.12 (t, J = 7.9 Hz, 1H), 1.89 − 1.80 (m, 4H), 1.79 (s, 2H), 1.78 − 1.63 (m, 6H), 1.64 − 1.44 (m, 4H), 0.93 − 0.82 (m, 3H). |
| Ex. 84 | 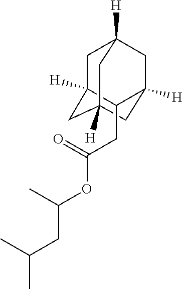 [SMILES: CC(C)CC(C)OC(=O)CC1[C@H]2C[C@@H]3C[C@@H](C[C@H]1C3)C2] | 4-methylpentan-2-yl 2- ((1R,3S,5r,7r)-adamantan- 2-yl)acetate | 278.22 | 1H NMR (300 MHz, DMSO-d6) δ 4.95 − 4.82 (m, 1H), 2.45 − 2.36 (m, 2H), 1.88 − 1.52 (m, 14H), 1.52 − 1.42 (m, 3H), 1.33 − 1.20 (m, 1H), 1.18 − 1.10 (m, 3H), 0.91 − 0.80 (m, 6H). |
| Ex. 85 | 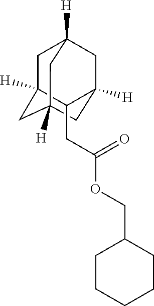 [SMILES: O=C(CC1[C@H]2C[C@@H]3C[C@@H](C[C@H]1C3)C2)OCC1CCCCC1] | cyclohexylmethyl 2- ((1R,3S,5r,7r)-adamantan- 2-yl)acetate | 290.22 | 1H NMR (300 MHz, DMSO-d6) δ 3.87 − 3.78 (m, 2H), 2.48 − 2.40 (m, 2H), 2.17 − 2.06 (m, 1H), 1.88 − 1.71 (m, 8H), 1.69 (s, 5H), 1.64 (s, 5H), 1.54 − 1.44 (m, 2H), 1.29 − 1.07 (m, 3H), 1.02 − 0.84 (m, 2H). |
| Ex. 86 | 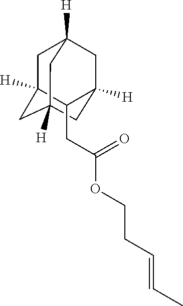 [SMILES: C/C=C/CCOC(=O)CC1[C@H]2C[C@@H]3C[C@@H](C[C@H]1C3)C2] | (E)-pent-3-en-1-yl 2- ((1R,3S,5r,7r)-adamantan- 2-yl)acetate | 262.19 | 1H NMR (300 MHz, DMSO-d6) δ 5.57 − 5.28 (m, 2H), 4.06 − 3.96 (m, 2H), 2.49 − 2.38 (m, 2H), 2.31 − 2.17 (m, 2H), 2.16 − 2.05 (m, 1H), 1.89 − 1.60 (m, 15H), 1.55 − 1.43 (m, 2H). |
| Ex. 87 | 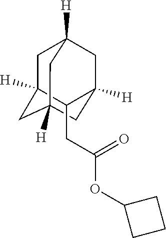 [SMILES: O=C(CC1[C@H]2C[C@@H]3C[C@@H](C[C@H]1C3)C2)OC1CCC1] | cyclobutyl 2- ((1R,3S,5r,7r)-adamantan- 2-yl)acetate | 248.18 | 1H NMR (300 MHz, DMSO-d6) δ 4.96 − 4.80 (m, 1H), 2.42 − 2.37 (m, 2H), 2.34 − 2.18 (m, 2H), 2.15 − 2.05 (m, 1H), 2.04 − 1.88 (m, 2H), 1.87 − 1.66 (m, 10H), 1.67 − 1.54 (m, 3H), 1.54 − 1.41 (m, 3H). |
| Ex. 88 | 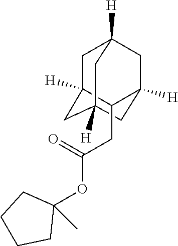 [SMILES: CC1(OC(=O)CC2[C@H]3C[C@@H]4C[C@@H](C[C@H]2C4)C3)CCCC1] | 1-methylcyclopentyl 2- ((1R,3S,5r,7r)-adamantan- 2-yl)acetate | 276.21 | 1H NMR (300 MHz, DMSO-d6) δ 2.38 − 2.29 (m, 2H), 2.14 − 1.94 (m, 3H), 1.89 − 1.71 (m, 7H), 1.69 (s, 2H), 1.64 (s, 5H), 1.62 − 1.55 (m, 3H), 1.49 (s, 4H). |

### Example 89. (1R,3S,5r,7r)-2-propoxyadamantane

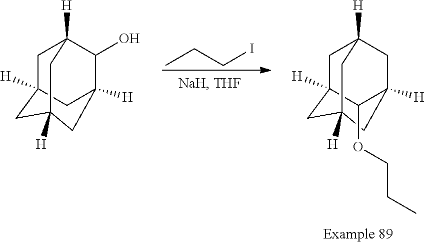

To a stirred mixture of (1r,3r,5r,7r)-adamantan-2-ol (500 mg, 3.29 mmol, 1.00 equiv.) in dry THF (100 mL) cooled to 0° C. is added the NaH (236 mg, 9.87 mmol, 3.00 equiv.). After 1 h, 1-iodopropane (1.67 g, 9.87 mmol, 3.00 equiv.) is added slowly. The resulting mixture is stirred and allowed to warm to 23° C. After 2 h, GC-MS indicates full consumption of starting material. The reaction is quenched with water/ice (100 mL) and aqueous NH₄Cl (3 M, 20 mL). The resulting biphasic mixture is extracted with Et₂O (3×50 mL) and the combined organic layers are washed with water (2×20 mL), dried over anhydrous Na₂SO₄, and concentrated under reduced pressure to afford the crude product as a yellow oil. The residue is purified by flash chromatography (silica gel, gradient 0% to 5% EtOAc in PE over 15 min) to afford (1R,3S,5r,7r)-2-propoxyadamantane (341.80 mg, 89.5%) as a yellow oil. GC-MS: (EI+, m/z, M⁺): 194.1 ¹H-NMR: (300 MHz, DMSO-d₆, ppm) δ 3.33 (s, 1H), 1.94 (s, 4H), 1.83-1.70 (m, 5H), 1.69-1.59 (m, 5H), 1.58-1.46 (m, 2H), 1.44 (s, 1H), 1.24 (s, 1H), 0.95-0.85 (m, 3H).

### Examples 90 to 93. The Compounds of Examples 7 to 19 are Prepared According the Procedure of Example 89, Substituting the Appropriate Alkyl Halide for the Iodopropane

| | | | GC-MS | |
|---|---|---|---|---|
| | | | (EI+, m/z, | |
| Ex. No. | Structure | Compound Name | M⁺) | ¹H-NMR |
| Ex. 90 | 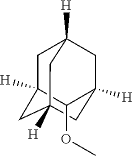 | (1R,3S,5r,7r)-2- methoxyadamantane | 166.14 | 1H NMR (300 MHz, DMSO-d6) δ 3.24 (s, 4H), 2.05 − 1.86 (m, 5H), 1.86 − 1.54 (m, 9H), 1.51 − 1.34 (m, 2H), 1.30 − 0.66 (m, 1H). |
| Ex. 91 | 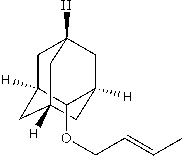 | (1R,3S,5r,7r)-2- (((E)-but-2-en-1- yl)oxy)adamantane | 206.17 | 1H NMR (300 MHz, DMSO-d6) δ 5.78 − 5.31 (m, 4H), 4.07 − 3.96 (m, 1H), 3.90-3.81 (m, 3H), 3.48-3.37 (m, 2H), 2.03 − 1.86 (m, 8H), 1.83- 1.70 (m, 8H), 1.69 − 1.50 (m, 14H), 1.48 − 1.32 (m, 4H). |
| Ex. 92 | 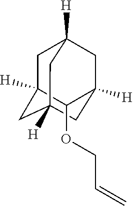 | (1R,3S,5r,7r)-2- (allyloxy)adamantane | 192.15 | 1H NMR (300 MHz, DMSO-d6) δ 6.01 − 5.83 (m, 1H), 5.34 − 5.20 (m, 1H), 5.17 − 5.05 (m, 1H), 4.00 − 3.92 (m, 2H), 3.48 − 3.40 (m, 1H), 2.01 − 1.93 (m, 4H), 1.85 − 1.71 (m, 4H), 1.70 − 1.57 (m, 4H), 1.49 − 1.37 (m, 2H). |
| Ex. 93 | 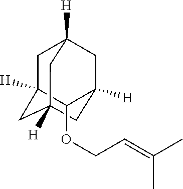 [SMILES: CC(C)=CCOC1[C@H]2C[C@@H]3C[C@@H](C[C@H]1C3)C2] | (1R,3S,5r,7r)-2-((3- methylbut-2-en-1- yl)oxy)adamantane | 220.18 | 1H NMR (400 MHz, DMSO-d6) δ 5.29 (ddt, J = 6.7, 5.1, 1.5 Hz, 1H), 3.91 (d, J = 6.7 Hz, 2H), 3.40 (d, J = 3.3 Hz, 1H), 1.95 (q, J = 4.2 Hz, 4H), 1.78 (dt, J = 11.4, 2.7 Hz, 3H), 1.74 − 1.68 (m, 4H), 1.68 − 1.58 (m, 7H), 1.45 − 1.37 (m, 2H). |

### Example 94. Olfactory Testing of Compounds

Compounds prepared according to the disclosure are studied for their olfactory qualities.

2-Ethyl-2-adamnantanol is obtained and its olfactory qualities are studied. Both the neat liquid compound and a 2% dilution of the compound in ethanol are examined by an experienced fragrance chemist (a master perfumer). It is found that the compound exhibits an odor characterized in the woods family, with prominent patchouli and camphoraceous notes.

2-Methyl-2-adamantol is obtained and its olfactory qualities are studied. A 10% dilution of the compound in ethanol is examined by an experienced fragrance chemist (a master perfumer). It is found that the compound exhibits an odor characterized by prominent camphor, fresh, aqueous and airy notes.

Following similar procedures, odor profile information is collected on the following additional compounds. The neat liquid compound and/or a 2% dilution of the compound in ethanol are examined by one or more experienced fragrance chemists (such as one or two junior perfumers and a master perfumer). In the below table, the term “uncharacterized” refers to a distinct odor, for which the evaluator did not provide a specific description.

| | Odor | |
|---|---|---|
| | Intensity | |
| Example | (0 to 10) | Odor Description |
| Example 1 | 5 | earthy, patchouli, pine absolute concentrated, |
| | | rosemary, terpenic, fir balsam |
| Example 2 | 0 | copper, bathroom cleaning spray, fresh, plastic, |
| | | oxidized/nutty |
| Example 3 | 5-6 | green, stemmy, woody, fruity, slightly |
| | | camphor, cypress, woody, slightly camphor |
| Example 4 | 7 | carrotseed, wet, earthy, dark. patchouli |
| Example 5 | 4 | fir balsam, anise, sweet |
| Example 6 | 3 | fresh, green, woody like popsicle stick of ice |
| | | cream, |
| Example 8 | 5 | acetone, slightly woody, cedarwood, creamy |
| | | in back, |
| Example 9 | 7 | camphor, patchouli, cumin. mostly camphor |
| | | covering the nuances |
| Example 10 | 3 | acid, woody patchouli, sweet, pine, fresh |
| Example 12 | 7 | camphor, patchouli, earthy, rosemary, fresh |
| Example 13 | 6 | woody, creamy, floral, milky, fresh |
| Example 14 | 1 | maybe slightly cedar or grapefruit peel, or |
| | | nothin |
| Example 15 | 6 | woody, carrotseed earthy, camphor, pine, |
| | | cashmeran in back, patchouli, but some |
| | | acid |
| Example 16 | 4 | banana, tropical, green, only a little artificial, |
| | | white floral jasmine sambac, slightly chlorine |
| | | after 2 min, , |
| Example 17 | 4 | fresh rosemary, cedar, fresh, only slightly |
| | | camphor, minty, |
| Example 18 | 6 | patchouli, patchoulol, wet, dusty, camphor, |
| | | wet soil, green |
| Example 19 | 4 | rosemary, cedar, like 041AM, earthy, |
| | | patchouli, |
| Example 20 | 3 | unscented burnt candle, wax |
| Example 22 | 3 | green olive, sweat, artificial fruit |
| Example 23 | 2 | orange/woody/plastic, uncharacterized |
| Example 24 | 2 | fresh, white cedar slightly, ethanol, aromatic |
| Example 26 | 2 | rubber, burnt garlic, camphor then those |
| | | disappear |
| Example 27 | 2 | slightly fishy, woody |
| Example 29 | 1 | slightly peel, uncharacterized |
| Example 30 | 1 | slightly fishy, |
| Example 31 | 3 | soft cedar, sweet floral, savory food, maybe |
| | | musk at end |
| Example 33 | 3 | strawberry, bright/sharp |
| Example 34 | 3 | aromatic, camphor, slightly sweaty like |
| | | borneol, patchoulol, green olive |
| Example 35 | 3 | technical acidic facet, radish sharp, |
| Example 36 | 3 | burnt, gas, lighting a gas stove, |
| Example 37 | 3 | sweat, olefin, sharp, body odor |
| Example 38 | 3 | first acetone, then green banana |
| Example 39 | 0 | uncharacterized |
| Example 40 | 3 | sprite soda (sparkly citrus), cedar |
| Example 41 | 2 | slightly cedar |
| Example 42 | 4 | cold coffee, cold fridge, fruity |
| Example 43 | 2 | vomit, soft |
| Example 44 | 4 | acetone, kitchen gas, then benzaldehyde, |
| | | almond anise heliotropin |
| Example 46 | 3 | cedar, sawdust, floral, soft |
| Example 47 | 3 | cedar, ambrox |
| Example 48 | 2 | ethanol, plastic |
| Example 49 | 3 | fruity, banana, parsnip |
| Example 50 | 3 | slightly sandalwood, rubbing alcohol |
| Example 52 | 3 | overripe, cedar wood, sweet |
| Example 53 | 4 | tropical fruit banana, woody, then honey, |
| | | patchouli |
| Example 54 | 4 | almond, paper, fresh, camphor, patchouli, |
| Example 55 | 4 | terpenic, dusty, raw carrot, wet soil, plastic |
| Example 56 | 5 | plastic, sandalwood, |
| Example 57 | 4 | patchouli, cedar, sweet, mirabelle plum, milky |
| Example 58 | 3 | plastic, acid, plastic like salicylate |
| Example 59 | 5 | acid, overripe, rubber tire, banana synthetic, |
| | | sprite, pineapple, chlorine |
| Example 60 | 3 | bright patchouli (but not earthy) |
| Example 61 | 2 | woody |
| Example 62 | 3 | woody, paper, sandalwood, plastic like |
| | | salicylate |
| Example 63 | 2 | aromatic, cleaner |
| Example 64 | 3 | dry fruit, cannabis, lime, terpenes |
| Example 65 | 4 | white cedar, dry earth |
| Example 66 | 2 | camphor, solvent |
| Example 67 | 1 | uncharacterized |
| Example 68 | 4 | dried garlic, dried onion, pine tree |
| Example 69 | 3 | artificial fruity, musky, cedar, waxy candy, |
| | | ambrox |
| Example 70 | 3 | camphor facet, cedar type atlas, medicinal |
| Example 71 | 2 | slightly raspberry, camphor |
| Example 72 | 2 | slightly camphor, uncharacterized |
| Example 73 | 3 | slightly painty floral like 18 expansions, |
| | | jasmine, fruity, strawberry |
| Example 74 | 2 | plastic, cedar, cis 3 salicylate, green |
| Example 75 | 3 | rubber tire, plastic |
| Example 76 | 4 | nice camphor, natural animallic vanilla |
| Example 77 | 4 | ether solvent, industrial, alcohol, slightly sweet |
| Example 78 | 3 | floral fruity pineapple |
| Example 79 | 3 | peach lactonic, fruity, paper, slightly |
| | | camphor |
| Example 80 | 2 | plastic |
| Example 81 | 3 | fruity, sweet, camphor |
| Example 82 | 3 | fruity peach lactonic |
| Example 83 | 3 | fruity, low intensity, slightly plastic, white |
| | | cedar |
| Example 84 | 1 | uncharacterized |
| Example 85 | 3 | cedar woody, slightly industrial solvent, floral, |
| | | green, jasmonal, slightly fruity pineapple facet |
| Example 86 | 4 | celery green jasmonal at first, then savory, |
| | | mushroom, white cedar at back |
| Example 87 | 4 | raw pumpkin, vegetal, floral, creamy, milky, |
| | | sweet |
| Example 88 | 3 | alcohol, paint, industrial |
| Example 89 | 5 | parsnip, leek, earthy, vegetable soup, tomato |
| | | paste, storebought tomato sauce, basil |
| Example 92 | 6 | unusual, thyme, olive, aromatic, mushroom, |
| | | meaty, tomato, sweet, campho rafter time, basil |

The Examples provided herein are exemplary only and are not intended to be limiting in any way to the various aspects and embodiments of the invention described herein.

1. A compound of Formula I: 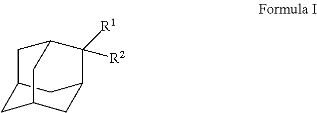
wherein:
R¹ is selected from OH, OR³, O(CO)R³, CN, COOH, COOR³, CH₂OH, CH₂OR³, C(O)R³, CH(R⁴)OR³, CH(R⁴)OH, CHO, CH₂COOH, and CH₂COOR³;
R² is H or optionally substituted C₁₋₆alkyl, C₂₋₆alkenyl, C₂₋₆alkynyl, or C₃₋₆ cycloalkyl;
R³ and R⁴ are each independently selected from C₁₋₆alkyl, C₂₋₆alkenyl, C₂₋₆alkynyl, C₃₋₇cycloalkyl, C₃₋₇cycloalkenyl, heterocycloalkyl, aryl, and heteroaryl, each of which is optionally substituted by one or more groups as defined herein;
wherein the adamantane core is optionally substituted by one or more groups as defined herein;
provided that the compound is not 2-methyl-2-adamantanol or 2-ethyl-2-admantanol; and
wherein the compound is not a compound wherein:
(a) R¹ is OH and R² is H, methyl, ethyl, propyl, isopropyl, cyclopropyl, n-butyl, tert-butyl, cyclobutyl, sec-butyl, isobutyl, cyclopropylmethyl, cyclopentyl, 1-ethylpropyl, n-pentyl, n-hexyl, cyclohexyl, vinyl, allyl, 1-propenyl, isopropenyl,1-propynyl, 2-propynyl, hydroxymethyl, aminomethyl, iodomethyl, cyanomethyl, 2-hydroxyethyl, methoxymethyl, aminoethyl, 3-hydroxypropyl, or 2-oxoethyl;
(b) R¹ is OCH₃ and R² is H, methyl, ethynyl, bromomethyl, aminomethyl, benzyl, allyl, 3-hydroxypropyl, isopropenyl, acetyl, carboxyl, CH₂COOH, CH₂CH₂COOH, 1-oxopropyl, or 3-oxobutyl;
(c) R¹ is O(CO)R³ and R³ is C₁₋₂alkyl, and R² is methyl, ethyl, ethynyl, vinyl, carboxy, acetyl, cyanomethyl, bromomethyl, CH₂COOH, 3-oxobutyl, or 3-(dimethylamino)propyl;
(d) R¹ is CH₂OH, and R² is methyl, aminomethyl, hydroxymethyl, allyl, 2-hydroxyethyl, vinyloxymethyl, (dimethylamino)methyl, 2-methoxyethyl, or CH₂O(CO)C(CH₃)═CH₂;
(e) R¹ is CH₂OR³, R² is methyl, and R³ is CH₂Cl, CH₂OCH═CH₃, CH₂O(CO)C(CH₃)═CH₂;
(f) R¹ is CH₂OCH═CH₃, and R² is CH₂OCH═CH₃ or CH₂OH;
(g) R¹ is COOH, and R² is methyl, 2-propenyl, aminomethyl, CH₂COOMe, CH₂CONHMe, CH₂CH₂COOH, CH₂CH₂CONHMe;
(h) R¹ is COOMe, and R² is methyl, 2-propenyl, aminomethyl, dicyanomethyl, 2-methoxyethyl, CH₂COOMe, CH₂CONHMe, CH₂CH₂COOH, CH₂CH₂CONHMe, or 2-phenylethyl; or
(i) R¹ is CN, and R² is methyl, hydroxymethyl, cyanomethyl, benzyl, 2-propenyl, n-butyl, aminohydroxymethyl, CH₂COOH, CH₂COOMe, CH₂COOEt, CH₂CH₂CH₂CO₂Et, 3-cyanopropyl, 2-methyl-2-buten-4-yl, 4-hydroxybutyl, 4-cyanobutyl, 4-chlorobutyl, 4-bromobutyl, or tetrazolylmethyl.

2. A compound of Formula I: 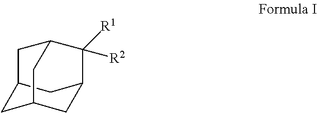
wherein:
R¹ is selected from OH, OR³, O(CO)R³, CN, COOH, COOR³, CH₂OH, CH₂OR³, C(O)R³, CH(R⁴)OR³, CH(R⁴)OH, CHO, CH₂COOH, and CH₂COOR³;
R² is H or optionally substituted C₁₋₆alkyl, C₂₋₆alkenyl, C₂₋₆alkynyl, or C₃₋₆cycloalkyl;
R³ and R⁴ are each independently selected from C₁₋₆alkyl, C₂₋₆alkenyl, C₂₋₆alkynyl, C₃₋₇cycloalkyl, C₃₋₇cycloalkenyl, heterocycloalkyl, aryl, and heteroaryl, each of which is optionally substituted by one or more groups as defined herein;
wherein the adamantane core is optionally substituted by one or more groups as defined herein,
for use as a flavor or fragrance ingredient.

3. A flavor composition and/or fragrance composition comprising a compound of Formula I: 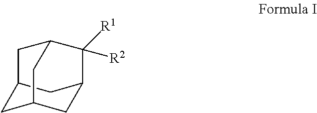
wherein:
R¹ is selected from OH, OR³, O(CO)R³, CN, COOH, COOR³, CH₂OH, CH₂OR³, C(O)R³, CH(R⁴)OR³, CH(R⁴)OH, CHO, CH₂COOH, and CH₂COOR³;
R² is H or optionally substituted C₁₋₆alkyl, C₂₋₆alkenyl, C₂₋₆alkynyl, or C₃₋₆cycloalkyl;
R³ and R⁴ are each independently selected from C₁₋₆alkyl, C₂₋₆alkenyl, C₂₋₆alkynyl, C₃₋₇cycloalkyl, C₃₋₇cycloalkenyl, heterocycloalkyl, aryl, and heteroaryl, each of which is optionally substituted by one or more groups as defined herein;
wherein the adamantane core is optionally substituted by one or more groups as defined herein, in admixture with one or more non-toxic, orally acceptable, pharmaceutically acceptable, cosmetically acceptable, or acceptable for a household product, carriers or excipients.

4. The composition of claim 3, wherein the compound of Formula I is:

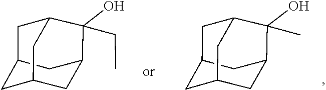

5. The composition of claim 3, wherein the compound of Formula I is a compound of Formula Ia: 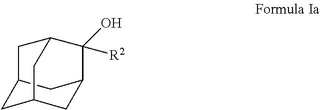
wherein R² is selected from methyl, ethyl, n-propyl, isopropyl, but-1-yl (n-butyl), but-2-yl (sec-butyl), 2-methyl-prop-1-yl (isobutyl), tert-butyl, pent-1-yl (n-pentyl), pent-2-yl (sec-pentyl), 3-methylbut-1-yl (isopentyl), pent-3-yl, hex-1-yl (n-hexyl), cyclopropyl, cyclobutyl, cyclopentyl, and cyclohexyl.

6. The composition of claim 3, wherein the compound of Formula I is a compound of Formula Ib: 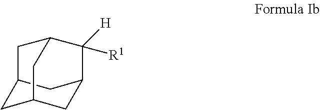
wherein R¹ is CH₂COOR³ and R³ is selected from methyl, ethyl, propyl, isopropyl, but-1-yl (n-butyl), but-2-yl (sec-butyl, e.g., (S)-but-2-yl), 2-methylprop-1-yl (isobutyl), tert-butyl, pent-1-yl (n-pentyl), pent-2-yl (e.g. (S)-pent-2-yl), 3-methyl-but-1-yl (isopentyl), 3-methylbut-2-yl, pent-3-yl, 2-methyl-but-1-yl, 2,2-dimethylpropyl (neopentyl), tert-pentyl, hex-1-yl (n-hexyl), hex-2-yl, hex-3-yl, 2-methylpent-1-yl, 4-methyl-pent-2-yl, and 3,3-dimethylbut-1-yl (neohexyl).

7. The composition of claim 3, wherein the compound of Formula I is a compound of Formula Ib: 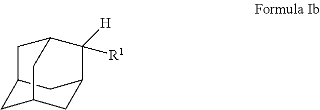
wherein R¹ is CH₂COOR³ and R³ is selected from but-3-en-1-yl, pent-2-en-1-yl (e.g., (E)-pent-2-en-1-yl), pent-3-en-1-yl (e.g., (E)-pent-3-en-1-yl), pent-4-en-2-yl, 2-methylbut-3-en-1-yl, 3-methylbut-2-en-1-yl, but-3-yn-1-yl, pent-4-yn-1-yl, and pent-4-yn-2-yl.

8. The composition of claim 3, wherein the compound of Formula I is a compound of Formula Ib: 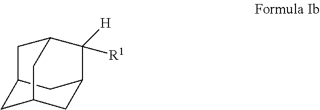
wherein R¹ is CH₂COOR³ and R³ is selected from cyclopropyl, cyclobutyl, 1-methylcyclobutyl, cyclopentyl, 3-methylcyclopentyl, cyclohexyl, 1-methylcyclopentyl, 2-methylcyclopent-1-yl, 2-methylcyclohexyl (e.g., (1S,2S)-2-methylcyclohexyl), 3-methylcyclohexyl, 4-methylcyclohexyl (e.g., (1R,2R)-4-methylcyclohexyl or (1S,4S)-4-methylcyclohexyl), and 2,2-dimethylcyclopent-1-yl, optionally wherein R³ is cycloheptyl.

9. The composition of claim 3, wherein the compound of Formula I is a compound of Formula Ib: 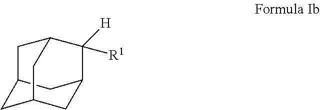
wherein R¹ is CH₂COOR³ and R³ is cyclopentene-4-yl, cyclohex-2-en-1-yl, or cyclohex-3-en-1-yl.

10. The composition of claim 3, wherein the compound of Formula I is a compound of Formula Ib: 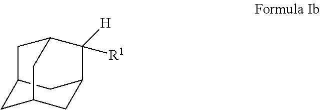
wherein R¹ is CH₂COOR³ and R³ is selected from cyclopropylmethyl, (1-methylcyclopropyl)methyl, cyclobutylmethyl, cyclopentylmethyl, 1-cyclopropylethyl, 2-cyclopropylethyl, 1-cyclobutylethyl, 2-cyclobutylethyl, 2-cyclopentylethyl, 2-cyclopentylethyl, 3-cyclopropylpropyl, cyclopent-3-en-1-ylmethyl, and cyclohexylmethyl.

11. The composition of claim 3, wherein the compound of Formula I is a compound of Formula Ib: 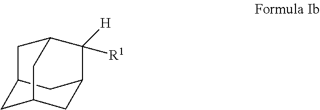
wherein R¹ is CH₂COOR³ and R³ is an optionally substituted bridged bicyclic C₃₋₆cycloalkyl or C₃₋₇cycloalkyl.

12. The composition of claim 11, wherein R³ is selected from bicyclo[2.2.1]hept-5-en-2-yl (e.g., (1S,4S)-bicyclo[2.2.1]hept-5-en-2-yl), pent-3-yn-1-yl, bicyclo[3.1.0]hexan-3-yl, and bicyclo[2.2.1]heptan-2-yl (e.g., (1R,2R,4S)-bicyclo[2.2.1]heptan-2-yl).

13. The composition of claim 3, wherein the compound of Formula I is a compound of Formula Ib 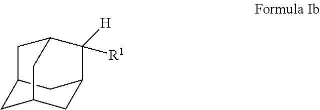
wherein R¹ is OR³ and R³ is selected from methyl, propyl, allyl, and 3-methylbut-2-en-1-yl.

14. The composition of claim 3, wherein the compound of Formula I is selected from one or more of the compounds of Example 1-93.

15. The composition of claim 3, wherein the composition further comprises one or more solvents.

16. The composition of claim 3, wherein the composition further comprises one or more other flavors or fragrances.

17. A product comprising the composition of claim 3, e.g., a product selected from the following: personal care products (e.g., a soap, skin cream or lotion, balm, shampoo, body wash, shower gel, hydrating cream, deodorant, antiperspirant, after-shave lotion, cologne, perfume, or other hair care or skin care product), sunscreens, insect repellants and insecticides, detergents, household cleaning agents (e.g., a surface cleaner, a metal cleaner, a wood cleaner, a glass cleaner, a body cleaner such as a soap, a dish-washing detergent, or a laundry detergent), air fresheners, room sprays, pomanders, candles, cosmetics (e.g., perfumes, colognes, nail polish, eye liner, mascara, lipstick, foundation, concealer, blush, bronzer, eye shadow, lip liner, lip balm), toilet waters, talcum powders, and pet litter.
